(19) United States
(12) Patent Application Publication
(10) Pub. No.: US 2026/0306564 A1
(43) Pub. Date: Oct. 8, 2026

(54) MIXTURE OF HMOS AND B. INFANTIS

(71) Applicant: SOCIÉTÉ DES PRODUITS NESTLÉ S.A., Vevey (CH)

(72) Inventors: Elizabeth FORBES-BLOM, La Conversion (CH); Dominick MAES, Villars-sur-glâne (CH); Mario NOTI, Vulliens (CH)

(21) Appl. No.: 19/483,520

(22) PCT Filed: May 15, 2024
(86) PCT No.: PCT/EP2024/063403

§ 371 Date: Nov. 11, 2025

(30) Foreign Application Priority Data
May 16, 2023 (EP) 23173614.1

(51) Int. Cl. A61K 35/745; A61K 9/00; A61K 31/702; A61P 11/06; A61P 31/14

(52) U.S. Cl. CPC A61K 35/745; A61K 9/0053; A61K 31/702; A61P 11/06; A61P 31/14

(57) ABSTRACT

The invention provides a composition for use in preventing and/or treating a viral infection in a subject, wherein the composition comprises Bifidobacterium longum subsp. infantis and a HMO mixture consisting of 2′-fucosyllactose (2FL), difucosyllactose (DFL), lacto-N-tetraose (LNT), 6′-sialyllactose (6SL), 3-sialyllactose (3SL), and 3-fucosyllactose (3FL). The invention also provides the composition for use in preventing and/or reducing the risk of allergen sensitisation, preventing and/or reducing the risk of developing a respiratory condition in a subject, and/or preventing and/or reducing the risk of developing asthma in a subject.

### FIELD OF THE INVENTION

The present invention relates to a composition comprising Bifidobacterium longum subsp. infantis and a HMO mixture consisting of 2′-fucosyllactose (2FL), difucosyllactose (DFL), lacto-N-tetraose (LNT), 6′-sialyllactose (6SL), 3′-sialyllactose (3SL), and 3-fucosyllactose (3FL). The invention also relates to applications of the composition in human health, for example, for i) preventing and/or treating a viral infection in a subject, ii) preventing and/or reducing the risk of allergen sensitisation, iii) preventing and/or reducing the risk of developing a respiratory condition in a subject, and/or iv) preventing and/or reducing the risk of developing asthma.

### BACKGROUND TO THE INVENTION

A viral infection occurs when the virus proliferates inside the host's cells and hence utilises the host's resources to promote its own multiplication. Viral infections can also interfere with the normal functioning of the host and may lead to more severe infection-related disorders, including long-term alterations in the immune system (such as inflammatory responses) and subsequent allergic or inflammatory diseases later in life.

Viral respiratory infections, such as respiratory syncytial virus (RSV), affect nearly 90% of children by the age of two (Karpinnen et al, Clin Microbiol Infect, 2016; 22; 208.e1-e6) and often lead to bronchiolitis, an inflammatory bronchial reaction in infants and young children (Pickles et al, J Pathol, 2015; 235; 266-276). In particular, severe RSV-induced bronchiolitis is a major cause of morbidity and mortality in infants globally (Nair et al, Lancet, 2010; 375; 9725; 2545-1555). Respiratory viruses primarily infect the airway epithelium. Higher viral loads have been associated with increased bronchiolitis severity and conversely, rapid viral load reduction in infants was associated with faster disease resolution (Pickles et al, J Pathol, 2015; 235; 266-276). It is well documented that infected and necrotic epithelial cells contribute to the airway obstruction and inflammation during RSV infection (Pickles et al, J Pathol, 2015; 235; 266-276) and as such epithelial cell sloughing is a feature of viral bronchiolitis and associated with disease severity (Johnson et al, Mod Pathol, 2007; 20; 108-119). Plasmacytoid dendritic cells (pDC) are known to be protective against pathology during RSV infection, and adaptive immune responses including CD4+ and CD8+ T cells are important in viral elimination from the respiratory tract (Openshaw et al, Annu Rev Immunol, 2017; 35; 501-532).

If the immune defense response to such viral respiratory infections is dysregulated, inflammatory granulocytes such as neutrophils along with CD4+ and CD8+ T cell responses can also lead to immunopathology following respiratory viral infection (Newton et al, Semin Immunopathol, 2016; 38; 471-482). Moreover, such uncontrolled inflammatory responses can also lead to pathological airway smooth muscle remodelling, a hallmark feature of asthma reported to commence in early life (O'Reilly et al, JACI, 2013; 131; 1024-1032) as well as playing a central role in the pathogenesis of chronic obstructive pulmonary disease (COPD; Yan F et al, J Transl Med, 2018; 16; 262-270). Thus, severe viral airway infections in early life represent an independent risk factor for subsequent development of respiratory diseases such as allergic airway disease (e.g. asthma; Feldman et al, Am J Respir Crit Care Med, 2015; 191; 34-44) and chronic obstructive pulmonary disease (Savran O et al. Int J Chron Obstruct Pulmon Dis. 2018; 13: 683-693) in later life.

Breastfeeding is a recognized factor that reduces severity of respiratory viral infection in infants either directly through milk bioactives (e.g. human milk oligosaccharides) or indirectly through microbiome mediated immune benefits.

Human milk oligosaccharides (HMOs) have become the subject of much interest in recent years due to their roles in numerous biological processes occurring in the human organism. Mammalian milk contains at least 130 of these complex oligosaccharides (Urashima et al, Milk Oligosaccharides, Nova Biomedical Books, New York, 2011, ISBN: 978-1-61122-831-1).

Infancy, especially the first weeks, 3 months, 6 months or 12 months of life is a critical period for the establishment of a balanced gut microbiota. It is known that the modulation of the gut microbiota during infancy and early childhood can prospectively have a significant influence in the future health status of the body. For example, the gut microbiome can have an influence on the development of a strong immune system later in life, as well as normal growth, and even on the development of obesity later in life. It is known that the mode of delivery can also affect the initial gut microbiota of infants: infants delivered by Caesarean section (C-section) have been shown to have a different gut microbiota compared to vaginally-delivered infants.

Bifidobacterium longum subsp. infantis (B. infantis) has been demonstrated to predominate in the gut microbiota of breastfed infants and to benefit the host by accelerating maturation of the immune response, balancing the immune system to suppress inflammation, improving intestinal barrier function, and increasing short-chain fatty acid (SCFA) production. Reduced abundance of Bifidobacterium species in infants and young children has been correlated to chronic diseases, including asthma and obesity, as well as to lower vaccine response. Researchers have postulated that loss of Bifidobacterium species in the infant gut in populations of developed countries is linked to increased incidence of allergic and autoimmune diseases.

It is known that probiotics, in particular from the Lactobacillus and Bifidobacterium genus, support protection against respiratory tract infections. The role of probiotics in viral respiratory tract infections was reviewed by Lehtoranta and co-workers (Lehtoranta et al, Eur J Clin Microbiol Infect Dis, 2014; 33; 1289-1302).

Increasing the abundance of Bifidobacterium species or specifically B. infantis in the gut microbial ecosystem through exogenous administration may be difficult to achieve, especially in formula-fed infants. Administration of a probiotic via nutritional compositions, such as infant formulae, typically aims to promote its engraftment in the gut microbiome so that it can elicit the desired benefits associated with said probiotic. The wide diversity of the microbial community in the gut makes probiotic engraftment challenging.

Due to the loss of Bifidobacterium species in the infant gut and low breast-feeding rates, there is a need to provide infants with both HMOs and HMO-utilizing bacteria such as B. longum subsp. infantis to support a healthy microbiome for long-term health.

Additionally, there are limited means to prevent or treat viral infections. There are limited numbers of effective antiviral drugs, for example drugs used to treat HIV and influenza, and the primary method to control viral disease is vaccination which is intended to prevent outbreaks by building immunity to a virus or a family of viruses.

Thus, there is a need for a composition to prevent and/or treat viral infections, and more specifically to prevent or treat viral infections of the respiratory tract.

### SUMMARY OF THE INVENTION

The present inventors have surprisingly found that synbiotic intervention (B. infantis in combination with a mix of human milk oligosaccharides) in early life provides protection from virus-induced bronchiolitis and promotes a sustained immune benefit into adulthood (as assessed by reduced susceptibility to pollution-enhanced allergic airway inflammation).

Specifically, the present inventors have shown that synbiotic interventions resulted in a rapid resolution of virus-induced lung inflammation and appropriate lung tissue remodeling upon clearance of the virus.

These findings support the use of the synbiotic in providing protection against and treatment of viral infections, in particular viral bronchiolitis, in early life and uncover functional benefits of the synbiotics to mount effective anti-viral immune responses associated with faster disease resolution. Given that viral respiratory tract infections in early life represent a major independent risk factor for subsequent asthma, recurrent wheeze and chronic obstructive pulmonary disease, dietary supplementation with the synbiotics may also prevent long-term complications associated with viral respiratory tract infections in early life. Accordingly, in a first aspect the present invention provides a composition for use in preventing and/or treating a viral infection in a subject, wherein the composition comprises Bifidobacterium longum subsp. infantis and a HMO mixture consisting of 2′-fucosyllactose (2FL), difucosyllactose (DFL), lacto-N-tetraose (LNT), 6′-sialyllactose (6SL), 3′-sialyllactose (3SL), and 3-fucosyllactose (3FL).

In a further aspect, the invention provides a composition for use in i) preventing and/or reducing the risk of allergen sensitisation in the respiratory tract and/or ii) preventing and/or reducing the risk of developing a respiratory condition in a subject, wherein the composition comprises Bifidobacterium longum subsp. infantis and a HMO mixture consisting of 2′-fucosyllactose (2FL), difucosyllactose (DFL), lacto-N-tetraose (LNT), 6′-sialyllactose (6SL), 3′-sialyllactose (3SL), and 3-fucosyllactose (3FL), and wherein the respiratory condition is a chronic inflammatory disease of the respiratory tract or an allergic respiratory tract disease.

In some embodiments, the composition is for use in preventing and/or reducing the risk of allergen sensitisation in the respiratory tract of a subject.

In some embodiments, the composition is for use in preventing and/or reducing the risk of developing a respiratory condition in a subject.

In a further aspect, the invention provides a composition for use in preventing and/or reducing the risk of developing asthma in a subject, wherein the composition comprises Bifidobacterium longum subsp. infantis and a HMO mixture consisting of 2′-fucosyllactose (2FL), difucosyllactose (DFL), lacto-N-tetraose (LNT), 6′-sialyllactose (6SL), 3′-sialyllactose (3SL), and 3-fucosyllactose (3FL).

In some embodiments, the Bifidobacterium longum subsp. infantis is Bifidobacterium longum subsp. infantis LMG 11588.

In some embodiments, the Bifidobacterium longum subsp. infantis is a strain having an Average Nucleotide Identity (ANI) of at least 99.9% to Bifidobacterium longum subsp. infantis LMG 11588.

In some embodiments, the HMO mixture consists essentially of:
i. 16 wt % to 69 wt % of 2FL, preferably 22 wt % to 59 wt %;
ii. 9 wt % to 24 wt % of LNT, preferably 12 wt % to 21 wt %;
iii. 2 wt % to 10 wt % of DFL, preferably 3 wt % to 8 wt %;
iv. 8 wt % to 26 wt % of 6SL and 3SL combined, preferably 11 wt % to 22 wt %;
and v. 8 wt % to 50 wt % of 3FL, preferably 11 wt % to 43 wt %.

In some embodiments, the composition is in the form of a nutritional composition.

In some embodiments, the nutritional composition is selected from an infant formula, a starter infant formula, a follow-on or follow-up formula, a baby food, an infant cereal composition, a growing-up-milk, a fortifier such as a human milk fortifier, or a supplement.

In some embodiments, the subject is an infant, a young child or a child. Suitably, the subject is an infant. Suitably, the subject is a young child. Suitably, the subject is a child.

In a further aspect, the invention provides the use of a composition according to the invention for the manufacture of a medicament for preventing and/or treating a viral infection in a subject.

In a further aspect, the invention provides the use of a composition according to the invention for the manufacture of a medicament for i) preventing and/or reducing the risk of allergen sensitisation in the respiratory tract and/or ii) preventing and/or reducing the risk of developing a respiratory condition in a subject, wherein the respiratory condition is a chronic inflammatory disease of the respiratory tract or an allergic respiratory tract disease.

In a further aspect, the invention provides the use of a composition according to the invention for the manufacture of a medicament for preventing and/or reducing the risk of developing asthma in a subject.

In a further aspect, the invention provides a method of preventing and/or treating a viral infection in a subject, the method comprising administering to the subject a composition according to the invention.

In a further aspect, the invention provides a method of i) preventing and/or reducing the risk of allergen sensitisation in the respiratory tract and/or ii) preventing and/or reducing the risk of developing a respiratory condition in a subject, the method comprising administering to the subject a composition according to the invention, wherein the respiratory condition is a chronic inflammatory disease of the respiratory tract or an allergic respiratory tract disease.

In a further aspect, the invention provides a method of preventing and/or reducing the risk of developing asthma in a subject, the method comprising administering to the subject a composition according to the invention.

### BRIEF DESCRIPTION OF THE FIGURES

FIG. 1: Schematic of model of early life viral airway infection and pollution enhanced allergic airway inflammation.

FIG. 2: Early life nutritional intervention with synbiotic (B. infantis+6HMOs) reduces virus-induced lung pathology.

FIGS. 3 and 4: Early life nutritional intervention with synbiotic (B. infantis+6HMOs) promotes sustained immune benefits into adulthood.

FIG. 5: Early life nutritional intervention with synbiotic (B. infantis+5HMOs) promotes anti-viral immune responses at peak of infection and reduces virus-induced lung inflammation.

FIG. 6: Synergy between the 7-HMOs in combination with B. infantis on the establishment of B. breve within faecal fermentation cultures.

### DETAILED DESCRIPTION OF THE INVENTION

### Definitions

As used herein, the following terms have the following meanings.

The term “subject” refers to an infant, young child, child, an infant small for gestational age (SGA) or a preterm infant.

The term “infant” means a child under the age of 12 months.

The expression “young child” means a child aged between one and three years, also called toddler.

The term “child” means a child aged between three and twelve years. Preferably, the term “child” means a child aged between three and six years.

A “preterm” or “premature” subject means an infant or young child who was not born at term.

Generally it refers to an infant or young child born prior 36 weeks of gestation.

By the expression “small for gestational age” or “SGA” it is referred to an infant or young child who is smaller in size than normal for their gestational age at birth, most commonly defined as a weight below the 10th percentile for the gestational age. In some embodiments, SGA may be associated with intrauterine growth restriction (IUGR), which refers to a condition in which a foetus is unable to achieve its potential size.

By the expression “low birth weight”, it should be understood as any body weight under 2500 g at birth.

The expression “nutritional composition” means a composition which nourishes a subject. This nutritional composition is usually to be taken orally or intravenously. It may include a lipid or fat source, a carbohydrate source and/or a protein source. In a particular embodiment the nutritional composition is a ready-to-drink composition such as a ready-to-drink formula.

In a particular embodiment, the nutritional composition of the present invention is a “synthetic nutritional composition”. The expression “synthetic nutritional composition” means a mixture obtained by chemical and/or biological means, which can be chemically identical to the mixture naturally occurring in mammalian milks (i.e. the synthetic nutritional composition is not breast milk).

The expression “infant formula” as used herein refers to a foodstuff intended for particular nutritional use by infants during the first months of life and satisfying by itself the nutritional requirements of this category of person (Article 2(c) of the European Commission Directive 91/321/EEC 2006/141/EC of 22 Dec. 2006 on infant formulae and follow-on formulae). It also refers to a nutritional composition intended for infants and as defined in Codex Alimentarius (Codex STAN 72-1981) and Infant Specialities (incl. Food for Special Medical Purpose).

The expression “infant formula” encompasses both “starter infant formula” and “follow-up formula” or “follow-on formula”.

A “follow-up formula” or “follow-on formula” is given from the 6th month onwards and includes “growing-up milk”. It constitutes the principal liquid element in the progressively diversified diet of this category of person.

The expression “growing-up milk” (or “GUM”) refers to a milk-based drink generally with added vitamins and minerals, that is intended for young children or children.

The expression “baby food” means a foodstuff intended for particular nutritional use by infants or young children during the first years of life.

The expression “infant cereal composition” means a foodstuff intended for particular nutritional use by infants or young children during the first years of life.

The term “fortifier” refers to liquid or solid nutritional compositions suitable for mixing with breast milk or infant formula.

The expression “weaning period” means the period during which the mother's milk is substituted by other food in the diet of an infant or young child.

The “mother's milk” should be understood as the breast milk or the colostrum of the mother.

An “oligosaccharide” is a saccharide polymer containing a small number (typically three to ten) of simple sugars (monosaccharides).

The term “HMO” or “HMOs” refers to human milk oligosaccharide(s). These carbohydrates are resistant to enzymatic hydrolysis by digestive enzymes (e.g. pancreatic and/or brush border), indicating that they may display functions not directly related to their caloric value. It has especially been illustrated that they play a vital role in the early development of infants and young children, such as the maturation of the immune system. Many different kinds of HMOs are found in the human milk. Each individual oligosaccharide is based on a combination of glucose, galactose, sialic acid (N-acetylneuraminic acid), fucose and/or N-acetylglucosamine with many and varied linkages between them, thus accounting for the enormous number of different oligosaccharides in human milk—over 130 such structures have been identified so far. Almost all of them have a lactose moiety at their reducing end while sialic acid and/or fucose (when present) occupy terminal positions at the non-reducing ends. The HMOs can be acidic (e.g. charged sialic acid containing oligosaccharide) or neutral (e.g. fucosylated oligosaccharide). Some examples of HMOs are the fucosylated oligosaccharides, the N-acetylated oligosaccharides and/or the sialylated oligosaccharides.

A “fucosylated oligosaccharide” is an oligosaccharide having a fucose residue. It has a neutral nature. Some examples are LNFP-I (lacto-N-fucopentaose 1), 2′-FL (2′ fucosyllactose), 3-FL (3-fucosyllactose).

The expressions “fucosylated oligosaccharides comprising an alpha-1,2-fucosyl-epitope” and “2-fucosylated oligosaccharides” encompass fucosylated oligosaccharides with a certain homology of form since they contain an alpha-1,2′-fucosyl-epitope, therefore a certain homology of function can be expected.

The expression “N-acetylated oligosaccharide(s)” encompasses both “N-acetyl-lactosamine” and “oligosaccharide(s) containing N-acetyl-lactosamine”. They are neutral oligosaccharides having an N-acetyl-lactosamine residue. Suitable examples are LNT (lacto-N-tetraose), para-lacto-N-neohexaose (para-LNnH), LNnT (lacto-N-neotetraose) and any combinations thereof. Other examples are lacto-N-hexaose, lacto-N-neohexaose, para-lacto-N-hexaose, para-lacto-N-neohexaose, lacto-N-octaose, lacto-N-neooctaose, iso-lacto-N-octaose, para-lacto-N-octaose and lacto-N-decaose.

A “sialylated oligosaccharide” is a charged sialic acid containing oligosaccharide, i.e. an oligosaccharide having a sialic acid residue. It has an acidic nature. Some examples are 3′-SL (3′-sialyllactose) and 6′-SL (6′-sialyllactose). The expressions “sialylated oligosaccharide” and “sialyllactose (SL)” can be used interchangeably. The trisaccharide sialyllactose consists of lactose at the reducing terminus and one sialic acid residue at the non-reducing end via an alpha-2,3 binding or alpha-2,6 binding, resulting in 3′-SL and 6′-SL, respectively.

A “precursor of HMO” is a key compound that intervenes in the manufacture of HMO, such as sialic acid and/or fucose.

The term “GOS” as used herein means “Galacto-oligosaccharide”. Galacto-oligosaccharides (GOS) as used herein typically consist of β-linked galactose moieties with galactose or glucose at the reducing end. Such GOS contains β-(1→2), β-(1→3), β-(1→4), or β-(1→6) linked galactose moieties and may have a degree of polymerization (DP) of 3-8 galactose units. The term GOS is therefore preferably referred to as oligosaccharide(s) comprising at least three galactose units, more preferably as oligosaccharide(s) comprising at least four galactose units, preferably having a degree of polymerization (DP) of 3-8 galactose units.

The nutritional composition of the present invention can be in solid form (e.g. powder) or in liquid form. The amount of the various ingredients (e.g. the oligosaccharides) can be expressed in g/100 g of composition on a dry weight basis when it is in a solid form, e.g. a powder, or as a concentration in g/L of the composition when it refers to a liquid form (this latter also encompasses liquid composition that may be obtained from a powder after reconstitution in a liquid such as milk, water, e.g. a reconstituted infant formula or a follow-on/follow-up formula or a growing-up milk or an infant cereal product or any other formulation designed for infant nutrition).

The expressions “infants/young children fed exclusively with human breast milk”, “infants or young children exclusively breast fed”, “exclusive breast fed infants or young children” and “breast-fed infants/young children” can be used interchangeably. They refer to infants or young children fed with a great majority (i.e. at least 90%, or at least 95%, or at least 99%) or all (100%) of nutrients and/or energy originating from human breast milk.

The expression “conventional nutritional composition” refers to standard synthetic nutritional compositions such as infant formula, follow-up milks or growing-up milks already found in the market.

The terms “microbial”, “microflora” and “microbiota” can be used interchangeably.

The expressions “microbiota in the gut”, “microbiota of the gut”, “gut microbiota” and “intestinal microbiota” can be used interchangeably.

By the expressions “preventing” or “prevention”, it is meant avoiding that a physical state, a condition or their consequences occurs and/or decreasing its incidence (i.e. reduction of the frequency). Prevention also encompasses delay or prevention of the onset of the symptoms of the disease, disorder or condition. Prevention may be absolute (such that no disease occurs) or may be effective only in some individuals or for a limited amount of time.

By the expressions “treating” or “treatment”, it is meant a decrease of the duration and/or of the severity of a physical state, a condition or their consequences (e.g. a decrease or elimination of symptoms of the condition). Treatment also encompasses to reduce, alleviate or eliminate one or more symptoms associated with the disease, disorder or condition which is being treated and/or to slow down, reduce or block the progression of the disease, disorder or condition which is being treated.

The prevention and/or the treatment of a physical state, a condition or their consequences can occur during the treatment (i.e. during the administration of the composition of the present invention, either immediately after the start of the administration or some time after, e.g. some days or weeks after the start). But it can also encompass the prevention and/or the treatment later in life. The term “later in life” encompasses the effect after the termination of the intervention or treatment. The effect “later in life” can be from 1 week to several months, or even years, for example from 2 to 4 weeks, from 2 to 6 weeks, from 2 to 8 weeks, from 1 to 6 months or from 2 to 12 months. Suitably, the effect “later in life” can be from 12 months to 12 years, such as from 2 years to 10 years, or from 4 years to 5 years, after the termination of the intervention or treatment. Suitably, the effect “later in life” lasts until the subject is at least 5 years of age, such as at least 10 years of age, at least 20 years of age or at least 30 years of age.

The term “prebiotic” means non-digestible carbohydrates that beneficially affect the host by selectively stimulating the growth and/or the activity of healthy bacteria such as bifidobacteria in the colon of humans (Gibson G R, Roberfroid M B. Dietary modulation of the human colonic microbiota: introducing the concept of prebiotics. J Nutr. 1995; 125:1401-12).

The term “probiotic” means microbial cell preparations or components of microbial cells with a beneficial effect on the health or well-being of the host. (Salminen S, Ouwehand A. Benno Y. et al. “Probiotics: how should they be defined” Trends Food Sci. Technol. 1999:10 107-10). The microbial cells are generally bacteria or yeasts.

The term “synbiotic” may refer to a component that contains both probiotics and prebiotics, or a live microbe and a substrate that is selectively utilized by the co-administered live microbe (see e.g. Swanson, K. S., et al., 2020. Nature Reviews Gastroenterology & Hepatology, 17(11), pp. 687-701).

The term “cfu” should be understood as colony-forming unit.

All percentages are by weight unless otherwise stated.

All weights expressed in g per 100 g of composition are dry weight unless otherwise stated.

The term “SCFA” means short chain fatty acid(s).

The expression “increasing SCFA production” means that the amount of systemic and/or colonic SCFA, is higher in an individual fed with the nutritional composition according to the present invention in comparison with a standard. The SCFA production may be measured by techniques known by the skilled person such as by Gas-Liquid Chromatography.

In the present context, the term “gastrointestinal tract” includes the mouth, pharynx, oesophagus, stomach, small intestine, large intestine, rectum and anus. The term “intestine” includes the small intestine, the large intestine and rectum.

In the present context, the term “respiratory tract” refers to the passage formed by the nose, nasal cavity, pharynx, larynx, trachea, bronchi and the lungs through which air passes during breathing.

The “Average Nucleotide Identity (ANI)” is a measure of nucleotide-level genomic similarity between the coding regions of two genomes. Average Nucleotide Identity can be assessed as describe here: Yoon S H, Ha S M, Lim J, Kwon S, Chun J. A large-scale evaluation of algorithms to calculate average nucleotide identity. Antonie Van Leeuwenhoek. 2017 October; 110(10):1281-1286. In the present embodiment the strain Bifidobacterium longum subsp. infantis LMG 11588 (also known as ATCC 17930) represents the reference genome to which a microbial genome is compared. An example of a microorganism genome that has at least 99.9% ANI with B. longum subsp. infantis LMG 11588 can be found in PATRIC (https://www.patricbrc.org), genome ID 1678.111. In one embodiment of the present invention, the Bifidobacterium longum subsp. infantis strain does not harbour potentially transferable antibiotic resistances.

The ANI of the shared genes between two strains is known to be a robust means to compare genetic relatedness among strains. Strains with ANI values of at least about 96% can be considered to belong to the same species (Konstantinidis and Tiedje, 2005, Proc Natl Acad Sci USA, 102(7):2567-72; and Goris et al., 2007, Int Syst Evol Microbiol. 57(Pt 1):81-91), while ANI values of at least about 99% indicate that the bacterial genomes belong to the same strain. The ANI between two bacterial genomes is calculated from pair-wise comparisons of all sequences shared between any two strains and can be determined, for example, using any of a number of publicly available ANI tools, including but not limited to OrthoANI with usearch (Yoon et al., 2017, Antonie van Leeuwenhoek 110:1281-1286); ANI Calculator, JSpecies (Richter and Rossello-Mora, 2009, Proc Natl Acad Sci USA 106:19126-19131); and JSpeciesWS (Richter et al., 2016, Bioinformatics 32:929-931). Other methods for determining the ANI of two genomes are known in the art (Konstantinidis, K. T. and Tiedje, 2005, J. M., Proc. Natl. Acad. Sci. U.S.A., 102: 2567-2572; and Varghese et al., 2015, Nucleic Acids Research, 43(14):6761-6771).

In addition, in the context of the invention, the terms “comprising” or “comprises” do not exclude other possible elements. The composition of the present invention, including the many embodiments described herein, can comprise, consist of, or consist essentially of the essential elements and limitations of the invention described herein, as well as any additional or optional ingredients, components, or limitations described herein or otherwise depending on the needs.

Any reference to prior art documents in this specification is not to be considered an admission that such prior art is widely known or forms part of the common general knowledge in the field.

The invention will now be described in further details. It is noted that the various aspects, features, examples and embodiments described in the present application may be compatible and/or combined together any combination thereof.

### Therapeutic Use

### Viral Infection

Viral respiratory infections, such as respiratory syncytial virus (RSV), affect nearly 90% of children by the age of two (Karpinnen et al, Clin Microbiol Infect, 2016; 22; 208.e1-e6). Such viral respiratory infections in infants and young children often lead to bronchiolitis, an inflammatory bronchial reaction in infants and young children (Pickles et al, J Pathol, 2015; 235; 266-276). Severe RSV-induced bronchiolitis is a major cause of morbidity and mortality in infants globally (Nair et al, Lancet, 2010; 375; 9725; 2545-1555).

Using a pneumonia Virus of Mice (PVM) model of human RSV infection, the present inventors have surprisingly found that synbiotic intervention (B. infantis in combination with a mix of human milk oligosaccharides) in early life provides protection from virus-induced bronchiolitis. These findings support the use of the synbiotic in providing protection against and treatment of viral infections, in particular viral bronchiolitis, in early life and uncover functional benefits of the synbiotics to mount effective anti-viral immune responses associated with faster disease resolution.

Thus, the composition of the invention is, in particular, effective for use in the treatment and/or prevention of a viral infection in a subject.

Accordingly, in a first aspect the present invention provides a composition for use in preventing and/or treating a viral infection in a subject, wherein the composition comprises Bifidobacterium longum subsp. infantis and a HMO mixture consisting of 2′-fucosyllactose (2FL), difucosyllactose (DFL), lacto-N-tetraose (LNT), 6′-sialyllactose (6SL), and 3′-sialyllactose (3SL), and optionally 3-fucosyllactose (3FL).

In a further aspect, the invention provides the use of a composition according to the invention for the manufacture of a medicament for preventing and/or treating a viral infection in a subject.

In a further aspect, the invention provides a method of preventing and/or treating a viral infection in a subject, the method comprising administering to the subject a composition according to the invention.

Accordingly, the composition of the invention is also effective for use in reducing the risk of contracting a viral infection in a subject and/or for use in reducing the symptoms associated with a viral infection in subject.

The viral infection may be a viral gastrointestinal infection or a viral respiratory tract infection. The viral gastrointestinal infection may be a viral intestinal infection or a viral stomach infection.

In a preferred embodiment, the viral infection is a viral respiratory tract infection. The viral respiratory tract infection may be a viral infection in the upper respiratory tract or in the lower respiratory tract. In a typical embodiment of the invention, the viral respiratory tract infection is caused by respiratory syncytial virus (RSV).

The disease associated with the viral infection will typically be common cold, influenza (flu), bronchitis, bronchiolitis, pneumonia, sore throat (pharyngitis), sinusitis, non-allergic rhinitis, severe acute respiratory syndrome (SARS), viral croup, otitis media, meningitis or diarrhoea. Typically, when the viral infection is in the respiratory tract, the disease associated with the respiratory tract infection is common cold, influenza (flu), bronchitis, bronchiolitis, pneumonia, sore throat (pharyngitis), sinusitis, non-allergic rhinitis, severe acute respiratory syndrome (SARS), viral croup or otitis media. Most often, the disease associated with the viral respiratory tract infection is common cold, influenza (flu), bronchitis, bronchiolitis or pneumonia.

Accordingly, in a preferred embodiment of the invention the composition of the invention is for use in treating and/or preventing a disease associated with a viral respiratory tract infection selected from the group consisting of common cold, influenza (flu), bronchitis, bronchiolitis and pneumonia. In a more preferred embodiment, the disease associated with the respiratory tract infection is selected from the group consisting of bronchiolitis and pneumonia, in particular RSV-induced bronchiolitis and/or pneumonia, i.e. bronchiolitis and/or pneumonia caused by RSV. In an even more preferred embodiment, the disease associated with the respiratory tract infection is bronchiolitis, in particular RSV-induced bronchiolitis.

The symptoms most often associated with the viral infection, and which may be reduced by the composition of the invention, are irritation in the lungs, congestion in the lungs, excessive mucus production, fever, cough, wheezing, breathlessness, abdominal cramps, diarrhoea or vomiting.

The above-mentioned infections may be caused by a variety of different viruses, including respiratory syncytial virus (RSV), parainfluenza virus (PIV), influenza virus such as influenza virus A (IVA) and/or influenza virus B (IVB), rhinovirus (RV), adenovirus (ADV), metapneumovirus (MPV), bocavirus (BoV), coronavirus (CoV), myxovirus, herpesvirus, enterovirus (EV), parachovirus (PeV) or a combination thereof.

The composition of the invention is particularly effective in treating, preventing, reducing the risk of contracting and/or reducing the symptoms of a viral infection caused by RSV. Thus, composition of the invention is particularly preferred for use in treating, preventing, reducing the risk of contracting and/or reducing the symptoms of RSV-induced bronchiolitis or RSV-induced pneumonia.

The composition of the invention is useful for treating and/or preventing viral infections, in particular respiratory tract infection in a human of any age. Thus, the human to be treated with the composition of the invention may be selected from the group consisting of 0 to <1 year (infants), 1 to <3 years (young children) and 3 to <6 years (children), including 3 to <5 years (pre-schoolers).

### Sustained Immune Benefit

Viral infections can also interfere with the normal functioning of the host and may lead to more severe infection-related disorders, including immunopathology following respiratory viral infection (Newton et al, Semin Immunopathol, 2016; 38; 471-482), such as long-term alterations in the immune system (e.g. inflammatory responses) and subsequent allergic or inflammatory diseases later in life. For example, uncontrolled inflammatory responses following viral infection of the respiratory tract can lead to pathological airway smooth muscle remodelling, a hallmark feature of asthma reported to commence in early life (O'Reilly et al, JACI, 2013; 131; 1024-1032) as well as playing a central role in the pathogenesis of chronic obstructive pulmonary disease (COPD; Yan F et al, J Transl Med, 2018; 16; 262-270). Thus, severe viral airway infections in early life represent a major independent risk factor for subsequent development of respiratory diseases such as allergic airway disease (e.g. asthma; Feldman et al, Am J Respir Crit Care Med, 2015; 191; 34-44) and chronic obstructive pulmonary disease (Savran O et al. Int J Chron Obstruct Pulmon Dis. 2018; 13: 683-693) in later life.

Thus, the composition of the invention is, in particular, effective for use in promoting a sustained immune benefit in a subject.

Accordingly, in a further aspect, the invention provides a composition for use in promoting a long-term immune benefit in a subject, wherein the composition comprises Bifidobacterium longum subsp. infantis and a HMO mixture consisting of 2′-fucosyllactose (2FL), difucosyllactose (DFL), lacto-N-tetraose (LNT), 6′-sialyllactose (6SL), and 3′-sialyllactose (3SL), and optionally 3-fucosyllactose (3FL.

In a further aspect, the invention provides the use of a composition according to the invention for the manufacture of a medicament for promoting a long-term immune benefit in a subject.

In a further aspect, the invention provides a method of promoting a long-term immune benefit in a subject, the method comprising administering to the subject a composition according to the invention.

Promoting a long-term immune benefit comprises:
i. promoting long-term respiratory health;
ii. preventing and/or reducing the risk of allergen sensitisation; and/or
iii. preventing and/or reducing the risk of developing a respiratory condition later in life.

Suitably, promoting a long-term immune benefit refers to promoting long-term respiratory health. Suitably, promoting a long-term immune benefit refers to preventing and/or reducing the risk of allergen sensitisation. Suitably, promoting a long-term immune benefit refers to preventing and/or reducing the risk of developing a respiratory condition later in life.

As used herein, the phrase “long-term” encompasses the effect after the termination of the intervention or treatment. The effect “long-term” can be from 1 week to several years, for example from 2 to 4 weeks, from 2 to 6 weeks, from 2 to 8 weeks, from 1 to 6 months, from 2 to 12 months, from 12 months to 12 years, such as from 2 years to 10 years, or from 4 years to 5 years, after the termination of the intervention or treatment. Hence, the effect “long term” may be present when the subject has reached an age of 3 years or more, preferably from 3 to 12 years, more preferably from 3 to 10 years, even more preferably from 3 to 8, most preferably from 3 to 6 years, in particular from 3 to 5 years or from 3 to 4 years. Suitably, the long-term benefit lasts until the subject is at least 5 years of age, such as at least 10 years of age, at least 20 years of age or at least 30 years of age.

Suitably, the composition of the invention may prevent a complication associated with a viral infection of the respiratory tract. Suitably, this effect may be the long-term prevention of a complication associated with a viral infection of the respiratory tract. Such complications may be those associated with the immune systems, such as the inflammatory response, and include immunopathology following respiratory viral infection, such as long-term alterations in the immune system (e.g. long-term alterations in inflammatory responses) and pathological airway smooth muscle remodelling. These complications may in turn predispose the subject to subsequent allergic or inflammatory diseases later in life, such as allergic respiratory diseases or chronic inflammatory diseases of the respiratory tract.

### Allergic Sensitization and Respiratory Conditions

Given that viral respiratory tract infections in early life represent a major independent risk factor for subsequent asthma, recurrent wheeze and chronic obstructive pulmonary disease later in life (Savran et al, Int J Chron Obstruct, 2015; 191; 34-44; and Feldman et al. 2015 Am J Respir Crit Care Med, 191; 34-44), the composition of the invention is also effective use in preventing and/or reducing the risk of developing respiratory conditions, such as chronic inflammatory diseases of the respiratory tract and allergic airway diseases.

Since viral infections, in particular infection with RSV, is associated with subsequent development of allergic airway diseases, such as asthma later in life (Feldman et al. 2015 Am J Respir Crit Care Med, 191; 34-44), the composition of the invention is also effective for use in preventing and/or reducing the risk of allergen sensitisation in the respiratory tract and/or developing an allergic respiratory tract disease in a subject.

Alongside B. infantis, Bifidobacterium breve is dominant in the faeces of breast-fed newborns and has a large body of evidence of immune modulating capability (Bozzi Cionci, N., et al., Nutrients, 2018, 10(11)). This evidence includes beneficial modulation of immune responses in the lung, including reduced airways hyperreactivity, T helper type 2 cell activation, eosinophilia and allergen specific antibody production (Hougee, S., et al., Int Arch Allergy Immunol, 2010, 151(2): 107-17).

The present inventors demonstrate a surprising synergistic effect of HMOs in combination with B. infantis on the establishment of B. breve within faecal fermentation cultures, thus connecting this synergistic effect to lung immunity and in particular i) preventing and/or treating a viral infection in a subject, ii) promoting a long-term immune benefit in a subject, iii) preventing and/or reducing the risk of allergen sensitization in the respiratory tract, iv) preventing and/or reducing the risk of developing a respiratory condition in a subject, and/or v) preventing and/or reducing the risk of developing asthma.

Accordingly, in a further aspect the invention provides a composition for use in i) preventing and/or reducing the risk of allergen sensitisation and/or ii) preventing and/or reducing the risk of developing a respiratory condition in a subject, wherein the composition comprises Bifidobacterium longum subsp. infantis and a HMO mixture consisting of 2′-fucosyllactose (2FL), difucosyllactose (DFL), lacto-N-tetraose (LNT), 6′-sialyllactose (6SL), and 3′-sialyllactose (3SL), and optionally 3-fucosyllactose (3FL).

In a further aspect, the invention provides a composition for use in preventing and/or reducing the risk of developing asthma in a subject, wherein the composition comprises Bifidobacterium longum subsp. infantis and a HMO mixture consisting of 2′-fucosyllactose (2FL), difucosyllactose (DFL), lacto-N-tetraose (LNT), 6′-sialyllactose (6SL), and 3′-sialyllactose (3SL), and optionally 3-fucosyllactose (3FL).

In a further aspect, the invention provides the use of a composition according to the invention for the manufacture of a medicament for i) preventing and/or reducing the risk of allergen sensitisation and/or ii) preventing and/or reducing the risk of developing a respiratory condition in a subject.

In a further aspect, the invention provides the use of a composition according to the invention for the manufacture of a medicament for preventing and/or reducing the risk of developing asthma in a subject.

In a further aspect, the invention provides a method of i) preventing and/or reducing the risk of allergen sensitisation and/or ii) preventing and/or reducing the risk of developing a respiratory condition in a subject, the method comprising administering to the subject a composition according to the invention.

In a further aspect, the invention provides a method of preventing and/or reducing the risk of developing asthma in a subject, the method comprising administering to the subject a composition according to the invention.

Respiratory conditions include chronic inflammatory diseases of the respiratory tract and allergic respiratory diseases.

Chronic inflammatory diseases of the respiratory tract include chronic obstructive pulmonary disease (COPD) and asthma, including allergic asthma and non-allergic asthma.

COPD is the term for a collection of lung diseases including chronic bronchitis, emphysema and chronic obstructive airways disease. People with COPD have difficulties breathing, primarily due to the narrowing of their airways.

Asthma is a chronic respiratory condition marked by inflammation and bronchospasm, causing difficulty in breathing. It is usually associated with an allergic reaction or other forms of hypersensitivity. Inflammation and narrowing of the small airways in the lungs cause asthma symptoms, which can be any combination of cough, wheeze, shortness of breath and chest tightness. Asthma often develops during childhood, particularly at the preschool stage (3 years to 5 years old).

Allergic respiratory tract diseases include recurrent wheeze and asthma, including allergic asthma.

For the i) preventing and/or reducing the risk of allergen sensitisation and/or ii) preventing and/or reducing the risk of developing a respiratory condition in a subject, the composition of the invention is preferably administered to a human having an age from 0 to <3 years, preferably from 0 to 2 years, more preferably from 0 to <1 year, such as from 0 to 6 months.

This, in turn, prevents and/or reduces the risk of developing a respiratory condition when the subject has reached an age of 3 years or more, preferably from 3 to 12 years, more preferably from 3 to 10 years, even more preferably from 3 to 8, most preferably from 3 to 6 years, in particular from 3 to 5 years or from 3 to 4 years.

Since viral infections, in particular infection with RSV, is often associated with bacterial co-infection (Thorburn et al, Thorax, 2006; 61(7); 611-615) or secondary infection (Sande et al, Nature Communications, 2019; 10; 2218), including antibiotic use, the composition of the invention is also effective for use in preventing or reducing the risk of a bacterial co-infection and/or a bacterial secondary infection associated with respiratory viral infection in a mammal, in particular a human. Pathogenic bacteria typically involved in co-infections or secondary infections include Staphylococcus aureus, Streptococcus pneumoniae and/or Haemophilus influenza.

### Composition

The present inventors have surprisingly found that the combination of Bifidobacterium longum subsp. infantis and a HMO mixture consisting of 2′-fucosyllactose (2FL), difucosyllactose (DFL), lacto-N-tetraose (LNT), 6′-sialyllactose (6SL), and 3′-sialyllactose (3SL), and optionally 3-fucosyllactose (3FL) in early life provides protection from virus-induced bronchiolitis and promotes a sustained immune benefit into adulthood (as assessed by reduced susceptibility to pollution-enhanced allergic airway inflammation).

As described above, the composition for use according to the invention comprises Bifidobacterium longum subsp. infantis and a HMO mixture consisting of 2′-fucosyllactose (2FL), difucosyllactose (DFL), lacto-N-tetraose (LNT), 6′-sialyllactose (6SL), and 3′-sialyllactose (3SL), and optionally 3-fucosyllactose (3FL).

The combination or composition of the invention may further comprise Bifidobacterium animalis subsp. lactis.

Accordingly, in a further aspect, the invention provides a composition comprising Bifidobacterium longum subsp. infantis, Bifidobacterium animalis subsp. lactis and a HMO mixture consisting of 2′-fucosyllactose (2FL), difucosyllactose (DFL), lacto-N-tetraose (LNT), 6′-sialyllactose (6SL), 3′-sialyllactose (3SL), lacto-N-neotetraose (LNnT) and optionally 3-fucosyllactose (3FL).

In a further aspect, the invention provides a combination comprising or consisting of Bifidobacterium longum subsp. infantis, Bifidobacterium animalis subsp. lactis and a HMO mixture consisting of 2′-fucosyllactose (2FL), difucosyllactose (DFL), lacto-N-tetraose (LNT), 6′-sialyllactose (6SL), 3′-sialyllactose (3SL), lacto-N-neotetraose (LNnT) and optionally 3-fucosyllactose (3FL). Preferably, the combination consists of Bifidobacterium longum subsp. infantis, Bifidobacterium animalis subsp. lactis and a HMO mixture consisting of 2′-fucosyllactose (2FL), difucosyllactose (DFL), lacto-N-tetraose (LNT), 6′-sialyllactose (6SL), 3′-sialyllactose (3SL), lacto-N-neotetraose (LNnT) and optionally 3-fucosyllactose (3FL).

Bifidobacterium longum subsp. infantis

Bifidobacterium longum is a bacterium of the Bifidobacterium genus which is present in the human gastrointestinal tract. In 2002, three previously distinct species of Bifidobacterium, B. infantis, B. longum, and B. suis, were unified into a single species named B. longum with the biotypes infantis, longum, and suis, respectively (Sakata, S., et al., 2002. International journal of systematic and evolutionary microbiology, 52(6), pp. 1945-1951).

Any suitable Bifidobacterium longum subsp. infantis strain may be used in the present invention. Such strains will be well-known to the skilled person. Suitable strains include Bifidobacterium longum subsp. infantis LMG 11588 (also known as Bifidobacterium longum subsp. infantis NCC3039 or Bifidobacterium longum subsp. infantis ATCC 17930) and Bifidobacterium longum subsp. infantis ATCC 15697 (also known as Bifidobacterium longum subsp. infantis NCC 3078).

The Bifidobacterium longum subsp. infantis may be a strain having at least 99% (suitably, at least 99.9%) ANI to Bifidobacterium longum subsp. infantis strain known to the skilled person.

Suitably, the Bifidobacterium longum subsp. infantis has at least 99% (suitably, at least 99.1%, at least 99.2%, at least 99.3%, at least 99.4%, at least 99.5%, at least 99.6%, at least 99.7%, at least 99.8%, at least 99.9%) ANI to Bifidobacterium longum subsp. infantis LMG 11588 (also known as Bifidobacterium longum subsp. infantis NCC3039 or Bifidobacterium longum subsp. infantis ATCC 17930). Preferably, the Bifidobacterium longum subsp. infantis has at least 99.9% ANI to Bifidobacterium longum subsp. infantis LMG 11588.

An example of a microorganism genome that has at least 99.9% ANI with B. longum subsp. infantis LMG 11588 can be found in PATRIC (https://www.patricbrc.org), genome ID 1678.111. Hence, suitably the B. longum subsp. infantis having the PATRIC genome ID 1678.111 may be used in the present invention.

Bifidobacterium longum subsp. infantis LMG 11588 is sold by the Belgian Coordinated Collections of Microorganisms (BCCM) under the LMG accession number LMG 11588.

Bifidobacterium longum subsp. infantis ATCC 15697 is sold by the American Type Culture Collection (ATCC) under the accession number ATCC 15697.

The composition or combination according to the invention may contain from 10³ to 10¹² cfu of Bifidobacterium longum subsp. infantis, more preferably between 10⁷ and 10¹² cfu such as between 10⁸ and 10¹⁰ cfu of Bifidobacterium longum subsp. infantis per g of composition or combination on a dry weight basis. Suitably, the Bifidobacterium longum subsp. infantis is administered to the subject in an amount of at least about 10⁶ cfu/day, at least about 10⁷ cfu/day, or at least about 10⁸ cfu/day. Suitably, the Bifidobacterium longum subsp. infantis is administered to the subject in an amount of about 10¹² cfu/day or less, about 10¹¹ cfu/day or less, or about 10¹⁰ cfu/day or less.

In one embodiment, the Bifidobacterium longum subsp. infantis is viable.

Bifidobacterium animalis subsp. lactis

Any suitable Bifidobacterium animalis subsp. lactis (B. lactis) strain may be used in the present invention. Such strains will be well-known to the skilled person. Suitable strains include Bifidobacterium animalis subsp. lactis CNCM 1-3446.

The Bifidobacterium animalis subsp. lactis may be a strain having at least 99% (suitably, at least 99.9%) ANI to a Bifidobacterium animalis subsp. lactis strain known to the skilled person.

Suitably, the Bifidobacterium animalis subsp. lactis has at least 99% (suitably, at least 99.1%, at least 99.2%, at least 99.3%, at least 99.4%, at least 99.5%, at least 99.6%, at least 99.7%, at least 99.8%, at least 99.9%) ANI to Bifidobacterium animalis subsp. lactis CNCM 1-3446. Preferably, the Bifidobacterium animalis subsp. lactis has at least 99.9% ANI to Bifidobacterium animalis subsp. lactis CNCM 1-3446.

Bifidobacterium lactis CNCM 1-3446 was deposited with the Collection Nationale de Cultures de Microorganismes (CNCM), Institut Pasteur (INSTITUT PASTEUR, 25 RUE DU DOCTEUR ROUX, F-75724 PARIS CEDEX 15, FRANCE) by NESTEC S. A. (NESTEC S. A., AVENUE NESTLE 55, CH-1800 VEVEY) according to the Budapest Treaty on 7 Jun. 2005 receiving the deposit number CNCM 1-3446.

The composition or combination according to the invention may contain from 10³ to 10¹² cfu of Bifidobacterium animalis subsp. lactis, more preferably between 10⁷ and 10¹² cfu such as between 10⁸ and 10¹⁰ cfu of Bifidobacterium animalis subsp. lactis per g of composition or combination on a dry weight basis. Suitably, the Bifidobacterium animalis subsp. lactis is administered to the subject in an amount of at least about 10⁶ cfu/day, at least about 10⁷ cfu/day, or at least about 10⁸ cfu/day. Suitably, the Bifidobacterium animalis subsp. lactis is administered to the subject in an amount of about 10¹² cfu/day or less, about 10¹¹ cfu/day or less, or about 10¹⁰ cfu/day or less.

The composition or combination according to the invention may contain from 10³ to 10¹² cfu of Bifidobacteria (e.g. a combination of Bifidobacterium longum subsp. infantis and Bifidobacterium animalis subsp. lactis) more preferably between 10⁷ and 10¹² cfu such as between 10⁸ and 10¹⁰ cfu of Bifidobacteria (e.g. of a combination of Bifidobacterium longum subsp. infantis and Bifidobacterium animalis subsp. lactis) per g of composition or combination on a dry weight basis. Suitably, the Bifidobacteria (e.g. a combination of Bifidobacterium longum subsp. infantis and Bifidobacterium animalis subsp. lactis) are administered to the subject in an amount of at least about 10⁶ cfu/day, at least about 10⁷ cfu/day, or at least about 10⁸ cfu/day. Suitably, the Bifidobacteria (e.g. a combination of Bifidobacterium longum subsp. infantis and Bifidobacterium animalis subsp. lactis) are administered to the subject in an amount of about 10¹² cfu/day or less, about 10¹¹ cfu/day or less, or about 10¹⁰ cfu/day or less.

In one embodiment, the Bifidobacterium animalis subsp. lactis is viable.

### HMO Mixture

The composition or combination of the invention comprises a HMO mixture.

In some embodiments, the HMO mixture consists of 2′-fucosyllactose (2FL), difucosyllactose (DFL), lacto-N-tetraose (LNT), 6′-sialyllactose (6SL), and 3′-sialyllactose (3SL).

In some embodiments, the HMO mixture consists of 2′-fucosyllactose (2FL), difucosyllactose (DFL), lacto-N-tetraose (LNT), 6′-sialyllactose (6SL), 3′-sialyllactose (3SL), and 3-fucosyllactose (3FL).

In some embodiments, the HMO mixture of the invention further comprises lacto-N-neotetraose (LNnT).

Accordingly, in some embodiments, the HMO mixture consists of 2′-fucosyllactose (2FL), difucosyllactose (DFL), lacto-N-tetraose (LNT), 6′-sialyllactose (6SL), 3′-sialyllactose (3SL), 3-fucosyllactose (3FL), and lacto-N-neotetraose (LNnT).

In some embodiments, the HMO mixture consists of 2′-fucosyllactose (2FL), difucosyllactose (DFL), lacto-N-tetraose (LNT), 6′-sialyllactose (6SL), 3′-sialyllactose (3SL), and lacto-N-neotetraose (LNnT).

In one embodiment, the HMO mixture comprises 2FL in an amount of from 16 wt % to 85 wt %. Suitably, the HMO mixture may comprise 2FL in an amount of from 31 wt % to 82 wt %, preferably from 41 wt % to 70 wt %. Suitably, the HMO mixture may comprise 2FL in an amount of from 16 wt % to 69 wt %, preferably from 22 wt % to 59 wt %.

In one embodiment, the HMO mixture comprises LNT in an amount of from 4 wt % to 40 wt %. Suitably, the HMO mixture may comprise LNT in an amount of from 10 wt % to 27 wt %, preferably from 14 wt % to 23 wt %. Suitably, the HMO mixture may comprise LNT in an amount of from 9 wt % to 24 wt %, preferably 12 wt % to 21 wt %.

In one embodiment, the HMO mixture comprises DFL in an amount of from 1 wt % to 14 wt %. Suitably, the HMO mixture may comprise DFL in an amount of from 4 wt % to 11 wt %, preferably from 6 wt % to 10 wt %. Suitably, the HMO mixture may comprise DFL in an amount of from 2 wt % to 10 wt %, preferably from 3 wt % to 8 wt %.

In one embodiment, the HMO mixture comprises 6SL and 3SL combined in an amount of from 7 wt % to 34 wt %. Suitably, the HMO mixture may comprise 6SL and 3SL combined in an amount of from 9 wt % to 34 wt %, preferably from 11 wt % to 29 wt %. Suitably, the HMO mixture may comprise 6SL and 3SL combined in an amount of from 8 wt % to 26 wt %, preferably from 11 wt % to 22 wt %.

In one embodiment, the HMO mixture comprises 3FL in an amount of from 10 wt % to 50 wt %. Suitably, the HMO mixture may comprise 3FL in an amount of from 18 wt % to 50 wt %, preferably from 11 wt % to 43 wt %.

In one embodiment, the HMO mixture comprises LNnT in an amount of from 6 wt % to 30 wt %. Suitably, the HMO mixture may comprise LNnT in an amount of from 6 wt % to 30 wt %, preferably from 7 wt % to 22 wt %. Suitably, the HMO mixture may comprise LNnT in an amount of from 3 wt % to 25 wt %, preferably from 5 wt % to 20 wt %.

In some embodiments, the HMO mixture consists essentially of:
i. 31 wt % to 82 wt % of 2FL;
ii. 10 wt % to 27 wt % of LNT;
iii. 4 wt % to 11 wt % of DFL; and
iv. 9 wt % to 34 wt % of 6SL and 3SL combined.

In some preferred embodiments, the HMO mixture consists essentially of:
i. 41 wt % to 70 wt % of 2FL;
ii. 14 wt % to 23 wt % of LNT;
iii. 6 wt % to 10 wt % of DFL; and
iv. 11 wt % to 29 wt % of 6SL and 3SL combined.

In some embodiments, the HMO mixture consists essentially of:
i. 16 wt % to 69 wt % of 2FL;
ii. 9 wt % to 24 wt % of LNT;
iii. 2 wt % to 10 wt % of DFL;
iv. 8 wt % to 26 wt % of 6SL and 3SL combined; and
v. 18 wt % to 50 wt % of 3FL.

In some preferred embodiments, the HMO mixture consists essentially of:
i. 22 wt % to 59 wt % of 2FL;
ii. 12 wt % to 21 wt % of LNT;
iii. 3 wt % to 8 wt % of DFL;
iv. 11 wt % to 22 wt % of 6SL and 3SL combined; and
v. 11 wt % to 43 wt % of 3FL.

In some embodiments, the HMO mixture consists essentially of:
i. 34 wt % to 85 wt % of 2FL;
ii. 10 wt % to 40 wt % of LNT;
iii. 4 wt % to 14 wt % of DFL;
iv. 9 wt % to 31 wt % of 6SL and 3SL combined; and
v. 6 wt % to 30 wt % of LNnT.

In some preferred embodiments, the HMO mixture consists essentially of:
i. 40 wt % to 71 wt % of 2FL;
ii. 12 wt % to 26 wt % of LNT;
iii. 5 wt % to 10 wt % of DFL; and
iv. 10 wt % to 28 wt % of 6SL and 3SL combined; and
v. 7 wt % to 22 wt % of LNnT.

In some embodiments, the HMO mixture consists essentially of:
i. 20 wt % to 60 wt % of 2FL;
ii. 4 wt % to 30 wt % of LNT;
iii. 1 wt % to 12 wt % of DFL;
iv. 7 wt % to 23 wt % of 6SL and 3SL combined;
v. 10 wt % to 50 wt % of 3FL; and
vi. 3 wt % to 25 wt % of LNnT.

In some preferred embodiments, the HMO mixture consists essentially of:
i. 22 wt % to 55 wt % of 2FL;
ii. 6 wt % to 20 wt % of LNT;
iii. 2 wt % to 8 wt % of DFL;
iv. 8 wt % to 22 wt % of 6SL and 3SL combined;
v. 13 wt % to 46 wt % of 3FL and
vi. 5 wt % to 20 wt % of LNnT.

When the composition or combination is in liquid form, the total HMO concentration is typically in the range from 0.5 to 10 g/L, preferably in the range from 1 to 7.5 g/L.

Specific examples of the concentration level of total HMO, when the composition or combination is in liquid form, include 1 to 5 g/L, 1 to 4 g/L, 2 to 5 g/L, 1 to 3 g/L or 2 to 4 g/L.

When the composition or combination is in solid form, the total HMO concentration is typically in the range from 0.35 to 7 wt % (g total HMO/100 g dry composition), preferably in the range from 0.35 to 5 wt %. Specific examples of the concentration level of total HMO, when the composition or combination is in dry form, include 0.5 to 3.5 wt % (g total HMO per 100 g dry composition), 0.5 to 2.5 wt %, 1 to 3.5 wt %, 0.5 to 2 wt % or 1 to 2.5 wt %.

### Subject

In some embodiments, the subject is a mammal, such as a human.

In some embodiments, the composition according to the invention is for use in infants, young children or children.

In one embodiment, the subject is an infant. In one embodiment, the subject is a young child. In one embodiment, the subject is a child.

The composition according to the invention is for use in infants or young children. It is particularly adapted for infants under 6 months of age.

In general, formula-fed infants have an underdeveloped immune system compared with adults and are more prone to viral infections than breastfed, and the younger the infant is, the less developed the immune system. Accordingly, the composition is particularly useful for preterm infants and/or low or very low birth weight infants, since these infants are even more vulnerable and prone to viral infections. In another particularly interesting embodiment, the composition is used in infants delivered via Caesarean section. Caesarean section born infants are born in a hospital in an environment having more pathogens against which the antibodies, transferred from the mother to the infant, are not effective against. Further, antibiotic administration is a recommended medical practice for C-section birth in order to prevent infection. Such interventions are potent disruptors of microbial communities (the mother's or the child's) and antibiotic treatment in early life is associated with an increased risk of developing immune mediated disorders later in life. Caesarean section born infants have a delayed and less optimal colonization of the large intestinal tract and are therefore also more prone to infections.

The infants, young children or children may be born term or preterm. In a particular embodiment, the nutritional composition of the invention is for use in infants, young children or children that were born preterm. Preterm infants may be at increased risk of poor nutrient utilization, impaired lean body mass growth, fat accumulation in the visceral area and metabolic disease later in life. Thus, in one embodiment the nutritional composition of the invention is for use in preterm infants.

In one embodiment, the subject is an infant or a young child that was born small for gestational age or low birth weight.

Infants or young children with low birth weight may or may not be preterm, and similarly, infants or young children who are small for gestational age may or may not be preterm.

The nutritional composition of the present invention may also be used in an infant or a young child that was born by C-section or that was vaginally delivered.

All infants and young children can benefit from the invention as all of them are or can be, at a certain age, susceptible to acquiring an unbalanced intestinal/gut microbiota.

In some advantageous embodiments of the invention, the nutritional composition in for use infants or young children having a fragile or unbalanced microbiota or dysbiosis of microbiota, such as preterm infants, infants born by Caesarean-section, infants born small for gestational age or with low birth weight, hospitalized infants/young children, infants/young children treated or having been treated by antibiotics and/or infants/young children suffering or having suffered from gut infection and/or gut inflammation.

It is indeed foreseen that the composition of the invention may be even more beneficial to infants born with possibly impaired gut microbiota or fragile infants/young children (such as prematurely born infants and/or infants born by C-section). It is also foreseen that the composition of the invention may be even more beneficial to infants/young children exhibiting intestinal disorders (such as diarrhea, infections or colic), especially after birth, for example, during the first 4 weeks after birth.

In embodiments of the invention, the infants born prematurely or born by caesarean section or born small for gestational age or with low birth weight, or exhibiting unbalanced or abnormal gut microbiota or suffering or having suffered from gut infection and/or gut inflammation, are targeted by the composition of the present invention, and especially when the infants are 0-6 months of age. Without being bound by the theory, it is believed that younger infants benefit even more from the composition of the invention, especially when the infants have (or are at risk of having) an unbalanced intestinal microbiota and/or have a fragile health condition (as exemplified by the conditions cited above).

The nutritional composition can be administered (or given or fed) at an age and for a period that depends on the needs.

In one embodiment, the infants or young children are 0-36 months of age, such as 0-12 months or 0-6 months of age. It is foreseen that the composition of the invention may be even more beneficial to infants just after birth (0-4 weeks or 0-8 weeks) as their intestinal tract may be more fragile.

The mammal to be treated is preferably a human being, but the mammal may also be non-human mammal, such as a non-human mammal selected from the group consisting of pig, cow, horse, dog, cat, goat, sheep and rabbit.

In other embodiments, the subject is a juvenile animal, preferably wherein the animal is a pet.

A pet may be a mammal such as dogs or cats, or rodents such as mice, rats, and guinea pigs, rabbits, etc. In some embodiments, the pet is a small dog breed.

The term “juvenile” may refer to an individual that has not yet reached adulthood.

In some embodiments the nutritional composition according to the invention can be for use before and/or during the weaning period.

In some embodiments the nutritional composition according to the invention is for use in a subject at risk and/or in need.

The subject at risk and/or in need may be bottle-fed and/or formula-fed.

In one embodiment the composition of the invention is given to the subject as a supplementary composition to the mother's milk. In some embodiments the subject receives the mother's milk during at least the first 2 weeks, first 1, 2, 4, or 6 months. In one embodiment the nutritional composition of the invention is given to the subject after such period of mother's nutrition, or is given together with such period of mother's milk nutrition. In another embodiment the composition is given to the subject as the sole or primary nutritional composition during at least one period of time, e.g. after the 1^{st}, 2^{nd} or 4^{th} month of life, during at least 1, 2, 4 or 6 months. In one embodiment the nutritional composition of the invention is a complete nutritional composition (fulfilling all or most of the nutritional needs of the subject). In another embodiment the nutrition composition of the invention is a supplement or a fortifier intended for example to supplement human milk or to supplement an infant formula or a follow-on formula.

### Nutritional Composition

In some embodiments, the composition of the invention is in the form of a nutritional composition.

The nutritional composition according to the invention can be for example an infant formula, a starter infant formula, a follow-on or follow-up formula, a growing-up milk, a baby food, an infant cereal composition, a fortifier such as a human milk fortifier, a supplement, a pet food, or a pet food supplement. In some particular embodiments, the composition of the invention is an infant formula, a fortifier or a supplement that may be intended for the first 4 or 6 months of age. In a preferred embodiment the nutritional composition of the invention is an infant formula.

In some other embodiments, the nutritional composition of the present invention is a fortifier. The fortifier can be a breast milk fortifier (e.g. a human milk fortifier) or a formula fortifier such as an infant formula fortifier or a follow-on/follow-up formula fortifier.

When the nutritional composition is a supplement, it can be provided in the form of unit doses. In such cases it is particularly useful to define the amount of oligosaccharides and probiotics in terms of daily dose to be administered to the infant, young child or child.

When the nutritional composition is a supplement, it may comprise the HMO mixture as described herein and B. infantis, and no other additional nutrient on top of the excipients necessary to obtain a stable nutritional composition.

The nutritional composition of the present invention can be in solid (e.g. powder), liquid or gelatinous form. In a specific embodiment the nutritional composition is a supplement, wherein the supplement is in powder form and provided in a sachet, preferably a sachet with 0.1 to 20 g per sachet, for example 1 to 10 g per sachet, or in the form of a syrup, preferably a syrup with a total solid concentration of 5 to 75 g/100 mL (5 to 75% (w/v)). When the supplement is in powder form, it may comprise a carrier. It is however preferred that the supplement is devoid of a carrier. When the supplement is in the form of a syrup, the components are preferably dissolved or suspended in water acidified with citrate.

In a particular embodiment the nutritional composition according to the invention is a hypoallergenic composition. In another particular embodiment the composition according to the invention is a hypoallergenic nutritional composition.

### Other Ingredients

The composition or combination according to the present invention may also comprise other types of oligosaccharide(s), polysaccharides and/or a fiber(s) and/or a precursor(s) thereof.

The other oligosaccharide and/or fiber and/or precursor thereof may be selected from the list comprising human milk oligosaccharides (HMOs), galacto-oligosaccharides (GOS), fructo-oligosaccharides (FOS), xylooligosaccharides (XOS), cello-oligosaccharides (COS), arabinoxylans, arabinans, xylans, inulin, polydextrose, beta-glucans, pectins and any combination thereof and any derived preparations thereof (e.g. partial hydrolysis). They may be in an amount between 0 and 10% by weight of composition. In a particular embodiment, the nutritional composition can also contain at least one BMO (bovine milk derived oligosaccharide).

Additional HMOs which may be included in the nutritional composition according to the present invention may be selected from the group consisting of lacto-N-fucopentaose (e.g. lacto-N-fucopentaose I, lacto-N-fucopentaose II, lacto-N-fucopentaose Ill, lacto-N-fucopentaose V), lacto-N-fucohexaose, lacto-N-difucohexaose I, fucosyllacto-N-hexaose, fucosyllacto-N-neohexaose, difucosyllacto-N-hexaose I, difucosyllacto-N-neohexaose II, para-lacto-N-neohexaose (para-LNnH), lacto-N-hexaose, lacto-N-neohexaose, para-lacto-N-hexaose, para-lacto-N-neohexaose, lacto-N-octaose, lacto-N-neooctaose, iso-lacto-N-octaose, para-lacto-N-octaose, lacto-N-decaose, and any combination thereof.

In some embodiments, the composition or combination according to the invention comprises at least one additional HMO.

In other embodiments, the composition or combination according to the present invention is devoid of any further HMOs. Thus, the prebiotics oligosaccharide mixture as described herein may be the only HMOs in the composition or combination of the invention.

The composition or combination of the present invention can further comprise at least one further probiotic (or probiotic strain), such as at least one further probiotic bacterial strain.

The probiotic microorganisms most commonly used are principally bacteria and yeasts of the following genera: Lactobacillus spp., Lacticaseibacillus spp, Limosilactobacillus spp, Streptococcus spp., Enterococcus spp., Bifidobacterium spp. and Saccharomyces spp.

In some particular embodiments, the probiotic is a probiotic bacterial strain. In some specific embodiments, it is particularly Bifidobacteria and/or Lactobacilli.

Suitable probiotic bacterial strains include Lactobacillus rhamnosus ATCC 53103 available from Valio Oy of Finland under the trademark LGG, Lactobacillus rhamnosus CGMCC 1.3724, Lactobacillus paracasei CNCM 1-2116, Lactobacillus johnsonii CNCM 1-1225, Streptococcus salivarius DSM 13084 sold by BLIS Technologies Limited of New Zealand under the designation K12, Bifidobacterium lactis sold inter alia by the Christian Hansen company of Denmark under the trademark Bb 12, B. longum CNCM 1-2618 (B. longum NCC2705), Bifidobacterium breve sold by Danisco under the trademark Bb-03, Bifidobacterium breve sold by Morinaga under the trade mark M-16V, Bifidobacterium infantis sold for example by Procter & Gamble Co. under the trademark Bifantis and Bifidobacterium breve sold by Institut Rosell (Lallemand) under the trademark R0070.

The composition or combination according to the invention may contain from 10e3 to 10e12 cfu of the at least one (further) probiotic strain, more preferably between 10e7 and 10e12 cfu such as between 10e8 and 10e10 cfu of probiotic strain per g of composition or combination on a dry weight basis.

In one embodiment, the probiotics are viable. In another embodiment, the probiotics are non-replicating or inactivated. There may be both viable probiotics and inactivated probiotics in some other embodiments. Probiotic components and metabolites can also be added.

The nutritional composition according to the invention generally contains a protein source. The protein can be in an amount of from 1.6 to 3 g per 100 kcal. In some embodiments, especially when the composition is intended for premature infants, the protein amount can be between 2.4 and 4 g/100 kcal or more than 3.6 g/100 kcal. In some other embodiments the protein amount can be below 2.0 g per 100 kcal, e.g. between 1.8 to 2 g/100 kcal, or in an amount below 1.8 g per 100 kcal.

Protein sources based on whey, casein and mixtures thereof may be used as well as protein sources based on soy. As far as whey proteins are concerned, the protein source may be based on acid whey or sweet whey or mixtures thereof and may include alpha-lactalbumin and beta-lactoglobulin in any desired proportions.

In some advantageous embodiments the protein source is whey predominant (i.e. more than 50% of proteins are coming from whey proteins, such as 60% or 70%).

The proteins may be intact or hydrolysed or a mixture of intact and hydrolysed proteins. By the term “intact” is meant that the main part of the proteins are intact, i.e. the molecular structure is not altered, for example at least 80% of the proteins are not altered, such as at least 85% of the proteins are not altered, preferably at least 90% of the proteins are not altered, even more preferably at least 95% of the proteins are not altered, such as at least 98% of the proteins are not altered. In a particular embodiment, 100% of the proteins are not altered.

The term “hydrolysed” means in the context of the present invention a protein which has been hydrolysed or broken down into its component amino acids. The proteins may be either fully or partially hydrolysed. It may be desirable to supply partially hydrolysed proteins (degree of hydrolysis between 2 and 20%), for example for infants or young children believed to be at risk of developing cow's milk allergy. If hydrolysed proteins are required, the hydrolysis process may be carried out as desired and as is known in the art. For example, whey protein hydrolysates may be prepared by enzymatically hydrolysing the whey fraction in one or more steps. If the whey fraction used as the starting material is substantially lactose free, it is found that the protein suffers much less lysine blockage during the hydrolysis process. This enables the extent of lysine blockage to be reduced from about 15% by weight of total lysine to less than about 10% by weight of lysine; for example about 7% by weight of lysine which greatly improves the nutritional quality of the protein source.

In an embodiment of the invention at least 70% of the proteins are hydrolysed, preferably at least 80% of the proteins are hydrolysed, such as at least 85% of the proteins are hydrolysed, even more preferably at least 90% of the proteins are hydrolysed, such as at least 95% of the proteins are hydrolysed, particularly at least 98% of the proteins are hydrolysed. In a particular embodiment, 100% of the proteins are hydrolysed.

In one particular embodiment the proteins of the nutritional composition are hydrolyzed, fully hydrolyzed or partially hydrolyzed. The degree of hydrolysis (DH) of the protein can be between 8 and 40, or between 20 and 60 or between 20 and 80 or more than 10, 20, 40, 60, 80 or 90.

The protein component can alternatively be replaced by a mixture or synthetic amino acid, for example for preterm or low birth weight infants.

In a particular embodiment, the nutritional composition or the growing-up milk according to the invention is a hypoallergenic composition. In another particular embodiment, the composition according to the invention is a hypoallergenic nutritional composition or growing-up milk.

The nutritional composition according to the present invention generally contains a carbohydrate source. This is particularly preferable in the case where the nutritional composition of the invention is an infant formula. In this case, any carbohydrate source conventionally found in infant formulae such as lactose, sucrose, saccharose, maltodextrin, starch and mixtures thereof may be used although one of the preferred sources of carbohydrates is lactose.

The nutritional composition according to the present invention generally contains a source of lipids. This is particularly relevant if the nutritional composition of the invention is an infant formula. In this case, the lipid source may be any lipid or fat which is suitable for use in infant formulae. Some suitable fat sources include palm oil, structured triglyceride oil, high oleic sunflower oil and high oleic safflower oil, medium-chain-triglyceride oil. The essential fatty acids linoleic and α-linolenic acid may also be added, as well small amounts of oils containing high quantities of preformed arachidonic acid and docosahexaenoic acid such as fish oils or microbial oils. The fat source may have a ratio of n-6 to n-3 fatty acids of about 5:1 to about 15:1; for example about 8:1 to about 10:1.

The nutritional composition of the invention may also contain all vitamins and minerals understood to be essential in the daily diet and in nutritionally significant amounts. Minimum requirements have been established for certain vitamins and minerals. Examples of minerals, vitamins and other nutrients optionally present in the composition of the invention include vitamin A, vitamin B1, vitamin B2, vitamin B6, vitamin B12, vitamin E, vitamin K, vitamin C, vitamin D, folic acid, inositol, niacin, biotin, pantothenic acid, choline, calcium, phosphorous, iodine, iron, magnesium, copper, zinc, manganese, chlorine, potassium, sodium, selenium, chromium, molybdenum, taurine, and L-carnitine. Minerals are usually added in salt form. The presence and amounts of specific minerals and other vitamins will vary depending on the intended population.

If necessary, the nutritional composition of the invention may contain emulsifiers and stabilisers such as soy, lecithin, citric acid esters of mono- and di-glycerides, and the like.

The nutritional composition of the invention may also contain other substances which may have a beneficial effect such as nucleotides, nucleosides, and the like.

The nutritional composition of the invention may also contain carotenoid(s). In some particular embodiments of the invention, the nutritional composition of the invention does not comprise any carotenoid.

### Manufacture of a Nutritional Composition

The nutritional composition according to the invention may be prepared in any suitable manner. A composition will now be described by way of example.

For example, a formula such as an infant formula may be prepared by blending together the protein source, the carbohydrate source and the fat source in appropriate proportions. If used, the emulsifiers may be included at this point. The vitamins and minerals may be added at this point but they are usually added later to avoid thermal degradation. Any lipophilic vitamins, emulsifiers and the like may be dissolved into the fat source prior to blending. Water, preferably water which has been subjected to reverse osmosis, may then be mixed in to form a liquid mixture. The temperature of the water is conveniently in the range between about 50° C. and about 80° C. to aid dispersal of the ingredients. Commercially available liquefiers may be used to form the liquid mixture.

The oligosaccharide(s) may be added at this stage, especially if the final product is to have a liquid form. If the final product is to be a powder, they may likewise be added at this stage if desired.

The liquid mixture is then homogenised, for example in two stages.

The liquid mixture may then be thermally treated to reduce bacterial loads, by rapidly heating the liquid mixture to a temperature in the range between about 80° C. and about 150° C. for a duration between about 5 seconds and about 5 minutes, for example. This may be carried out by means of steam injection, an autoclave or a heat exchanger, for example a plate heat exchanger.

Then, the liquid mixture may be cooled to between about 60° C. and about 85° C. for example by flash cooling. The liquid mixture may then be again homogenised, for example in two stages between about 10 MPa and about 30 MPa in the first stage and between about 2 MPa and about 10 MPa in the second stage. The homogenised mixture may then be further cooled to add any heat sensitive components, such as vitamins and minerals. The pH and solids content of the homogenised mixture are conveniently adjusted at this point.

If the final product is to be a powder, the homogenised mixture is transferred to a suitable drying apparatus such as a spray dryer or freeze dryer and converted to powder. The powder should have a moisture content of less than about 5% by weight. The oligosaccharide(s) may also or alternatively be added at this stage by dry-mixing or by blending them in a syrup form of crystals, along with the probiotic strain(s), and the mixture is spray-dried or freeze-dried.

If a liquid composition is preferred, the homogenised mixture may be sterilised then aseptically filled into suitable containers or may be first filled into the containers and then retorted.

In another embodiment, the composition of the invention may be a supplement. The supplement may be in the form of tablets, capsules, pastilles or a liquid for example. The supplement may further contain protective hydrocolloids (such as gums, proteins, modified starches), binders, film forming agents, encapsulating agents/materials, wall/shell materials, matrix compounds, coatings, emulsifiers, surface active agents, solubilizing agents (oils, fats, waxes, lecithins etc.), adsorbents, carriers, fillers, co-compounds, dispersing agents, wetting agents, processing aids (solvents), flowing agents, taste masking agents, weighting agents, jellifying agents and gel forming agents. The supplement may also contain conventional pharmaceutical additives and adjuvants, excipients and diluents, including, but not limited to, water, gelatine of any origin, vegetable gums, lignin-sulfonate, talc, sugars, starch, gum arabic, vegetable oils, polyalkylene glycols, flavouring agents, preservatives, stabilizers, emulsifying agents, buffers, lubricants, colorants, wetting agents, fillers, and the like.

Further, the supplement may contain an organic or inorganic carrier material suitable for oral or parenteral administration as well as vitamins, minerals trace elements and other micronutrients in accordance with the recommendations of Government bodies such as the USRDA.

This disclosure is not limited by the exemplary methods and materials disclosed herein, and any methods and materials similar or equivalent to those described herein can be used in the practice or testing of embodiments of this disclosure. Numeric ranges are inclusive of the numbers defining the range. Unless otherwise indicated, any nucleic acid sequences are written left to right in 5′ to 3′ orientation; amino acid sequences are written left to right in amino to carboxy orientation, respectively.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within this disclosure. The upper and lower limits of these smaller ranges may independently be included or excluded in the range, and each range where either, neither or both limits are included in the smaller ranges is also encompassed within this disclosure, subject to any specifically excluded limit in the stated range. Where the stated range includes one or both of the limits, ranges excluding either or both of those included limits are also included in this disclosure.

It must be noted that as used herein and in the appended claims, the singular forms “a”, “an”, and “the” include plural referents unless the context clearly dictates otherwise.

The terms “comprising”, “comprises” and “comprised of’ as used herein are synonymous with “including”, “includes” or “containing”, “contains”, and are inclusive or open-ended and do not exclude additional, non-recited members, elements or method steps. The terms “comprising”, “comprises” and “comprised of’ also include the term “consisting of’.

The practice of the present invention will employ, unless otherwise indicated, conventional techniques of chemistry, biochemistry, molecular biology, microbiology and immunology, which are within the capabilities of a person of ordinary skill in the art. Such techniques are explained in the literature. See, for example, Sambrook, J., Fritsch, E. F. and Maniatis, T. (1989) Molecular Cloning: A Laboratory Manual, 2nd Edition, Cold Spring Harbor Laboratory Press; Ausubel, F. M. et al. (1995 and periodic supplements) Current Protocols in Molecular Biology, Ch. 9, 13 and 16, John Wiley & Sons; Roe, B., Crabtree, J. and Kahn, A. (1996) DNA Isolation and Sequencing: Essential Techniques, John Wiley & Sons; Polak, J. M. and McGee, J. O'D. (1990) In Situ Hybridization: Principles and Practice, Oxford University Press; Gait, M. J. (1984) Oligonucleotide Synthesis: A Practical Approach, IRL Press; and Lilley, D. M. and Dahlberg, J. E. (1992) Methods in Enzymology: DNA Structures Part A: Synthesis and Physical Analysis of DNA, Academic Press. Each of these general texts is herein incorporated by reference.

The publications discussed herein are provided solely for their disclosure prior to the filing date of the present application. Nothing herein is to be construed as an admission that such publications constitute prior art to the claims appended hereto.

The invention will now be further described by way of Examples, which are meant to serve to assist one of ordinary skill in the art in carrying out the invention and are not intended in any way to limit the scope of the invention.

### EXAMPLES

### Example 1—Effects on Early Life Viral Airway Infection and Pollution Enhanced Allergic Airway Inflammation

### Detailed Trial Design

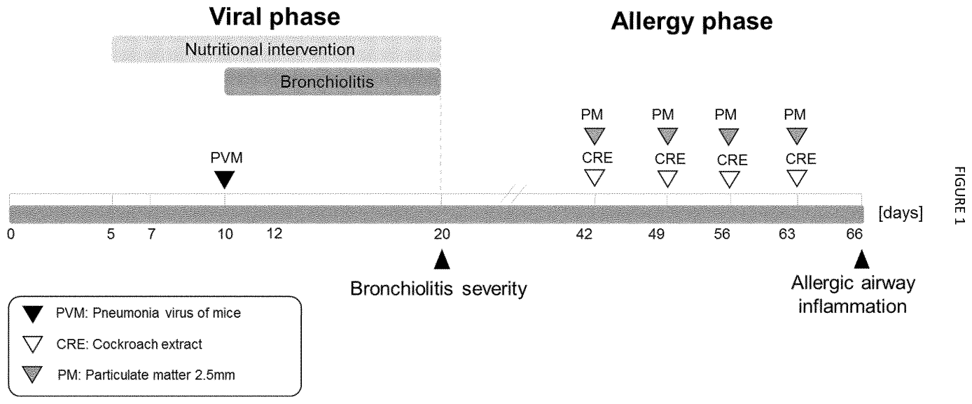

FIG. 1 shows a schematic of the model of early life viral airway infection and pollution enhanced allergic airway inflammation. The trial design is described in more detail below.

WT C57BL/6 breeders were fed either a control fiber or low fiber diet, starting 3 weeks prior to gestation. Offspring of these breeders were fed the corresponding diet of their mothers throughout the experiment, i.e. from post-natal day (PND) 0 to PND66.

At PND5, litters of low fiber diet fed animals were randomly assigned to the different experimental groups (n=14-16/group). Mice were fed once daily with different combinations of nutritional ingredients (6HMOs; B. infantis LMG11588; B. infantis LMG11588+6HMOs) via intra-gastric (i.g.) gavage of 50 μl of ingredients in a saline solution ending PND20. Control groups were fed with saline solution only.

At PND10, all animals (n=13-16/group) were infected intra-nasally (i.n.) with 10 PFU of PVM (PVM, J3666) in a total volume of 40 μl in a saline solution (20 μl/nostril) to induce bronchiolitis. The solution containing the virus was delivered as droplets on the nostrils using P20 pipettes. Isoflurane anesthetized mice were hold in supine position to make sure the solution was properly inhaled.

Animals (n=7-8/group) were sacrificed at PND20 (at peak immunopathology of bronchiolitis) to measure virus induced lung inflammation and associated pathology.

Starting from PND42, animals that recovered from PVM-infection (n=7-8/group) were sensitized i.n. with cockroach allergen extracts (CRE, 1 mg/application) in combination with particulate matter PM2.5 (10 mg) once a week ending PND63 (i.e. on PND42, PND49, PND56, PND63) to induce pollution-enhanced allergic airway inflammation.

Animals were either sacrificed on PND20 (N=7-8/group, peak immunopathology bronchiolitis) to test impact of nutritional interventions to promote protective immunity and on PND66 (N=7-8/group) to assess susceptibility to pollution-enhanced allergic airway inflammation post viral airway infection.

### Probiotic Dose

10⁶ CFU/day.

### HMO Dose

A concentration of 4 mg/day and application of a mix of 6HMOs (2FL/DFL, LNT, 3SL, 6SL, 3FL) has been selected based on most recent recommendations for HMOs in infant formulae (Stage 1: 1.8-2 g/L/day in the first 6 months).

Calculations were based on 1-week old infant consuming 500 mL of breast milk/day and 10 day old mouse pup consuming up to 2 mL of milk/day (Source: contemporary topics in laboratory animal science/American Association for Laboratory Animal Science 43(3):50-3).

The ratio between HMO ingredients is inspired on breast milk composition: 2FL/DFL ingredient 53%; LNT ingredient 19%; 6SL ingredient 8%, 3SL ingredient 6.2%; 3FL ingredient 13.8%.

In some experiments a concentration of 4 mg/day and application of a blend of 5HMOs (2FL, DFL, LNT, 3SL, 6SL) has been selected based on most recent recommendations for HMOs in infant formulae (Stage 1: 1.8-2 g/L/day in the first 6 months). Ratios used for 5HMO blend: 2FL/DFL ingredient 65.70%; LNT ingredient 23.40%; 6SL ingredient 9%, 3SL ingredient 2%.

### Low Fiber Diet

WT C57BL/6 mice efficiently clear the PVM virus. Such mild infections are not associated with altered lung tissue remodeling that in infants predispose to allergic airway inflammation later in life. To increase susceptibility to PVM infection and associated risk of allergic airway inflammation in adulthood, dams and corresponding pups were fed a low fiber diet throughout the experiment (susceptible groups; Trompette, Nat. Med, 2014). Animals fed a normal fiber diet were used as protected controls (protected group).

Statistical Analysis Statistical significance was determined using a non-parametric, two-was ANOVA followed by Bonferroni post-hoc testing. Results were considered significant at P≤0.05. *P≤0.05; ** P≤0.01; *** P≤0.001. All statistical analyses were performed using Prism GraphPad Software.

### Results

### PVM Infection Model

Pneumonia Virus of Mice (PVM) is related to the human respiratory syncytial virus (RSV) pathogen that affects >90% of children by the age of two years. PVM has been used to study respiratory virus replication and the ensuing inflammatory response as a component of a natural host-pathogen relationship. As such, PVM infections in mice reproduce many of the clinical and pathologic features of the more severe forms of RSV infections in infants—that if not properly controlled have long-lasting consequences on lung function and predispose to the onset of allergic airway responses later in life (Dyer K D, Viruses, 2012). The highly pathogenic PVM strain J3666 has been selected for these studies to conform to human pathophysiology of RSV infections in infants.

Severe bronchiolitis in early life is associated with mucus hypersecretion and airway epithelial cell sloughing. Dead epithelial cells together with a viscous exudate can form dense plugs in the bronchioalveolar lumen that impede breathing. Controlled mucus secretion and epithelial cell death are beneficial defense mechanisms limiting viral spread. These responses need to be tightly regulated upon clearance of virus to avoid chronicity and pathological tissue remodeling.

Pathological hallmarks of a severe viral lower respiratory infection include airway epithelial cell (AEC) sloughing, mucus hypersecretion and airway smooth muscle (ASM) remodelling. AEC detachment is a feature of viral bronchiolitis and is associated with disease severity and viral load.

AEC sloughing was quantified by measuring the length of sloughed airway epithelium and expressing this as a percentage as basement membrane length of airway in at least five airways per mouse. Mucus hyper-secretion and pathological lung tissue remodeling as other hallmarks of severe bronchiolitis were assessed by quantification of Muc5ac positive AECs, and ASM remodeling was assessed by area/airway circumference, respectively (Lynch J P, JEM 2018).

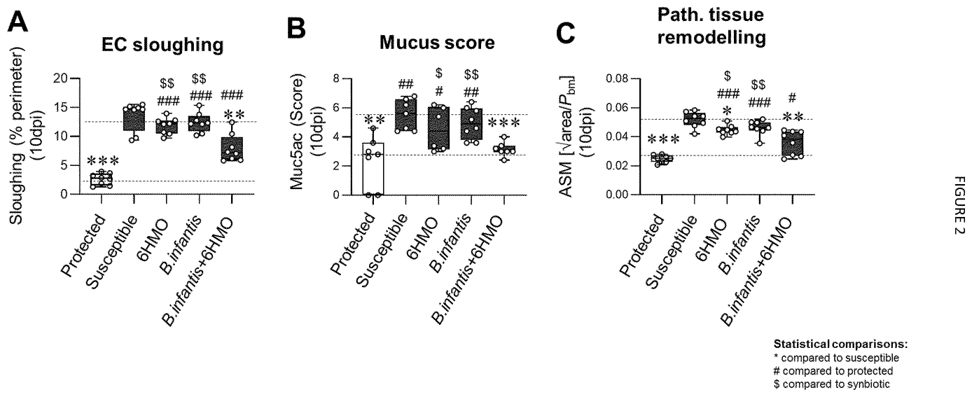

Early life nutritional intervention with synbiotic (B. infantis+6HMOs) resulted in a significant reduction in infection mediated airway epithelial cell (AEC) sloughing (FIG. 2A), mucus secretion (FIG. 2B) and pathological tissue remodeling (FIG. 2C) compared to placebo treated controls. Strikingly, synbiotic intervention showed superior efficacy over prebiotic (6HMO) or probiotic (B. infantis) interventions alone in reducing infection-mediated AEC sloughing, mucus secretion and pathological airway remodeling.

To assess whether early life nutritional supplementation with the synbiotic, besides eliciting an effective anti-viral immune response, results in controlled resolution of lung inflammation upon viral clearance, numbers of neutrophils and/or eosinophils in the airways of PVM infected mice were assessed by flow cytometry. Importantly, early life supplementation with the synbiotic resulted in controlled resolution of lung inflammation upon clearance of virus as assessed by significantly reduced numbers of granulocytes (neutrophils and eosinophils) in the airways in response to PVM infection.

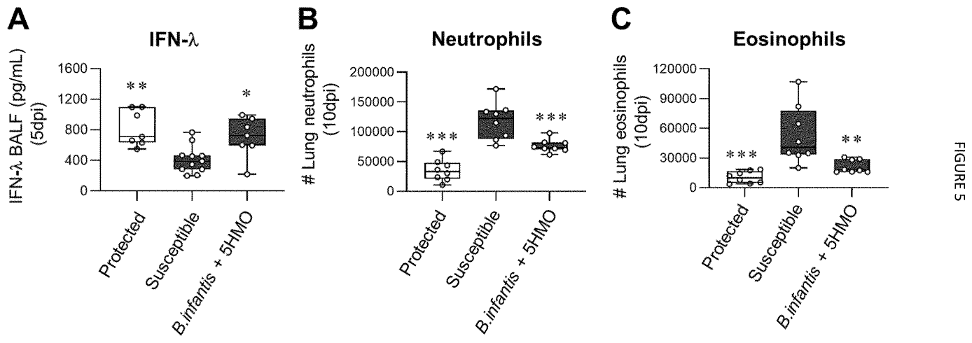

Early life nutritional intervention with a second synbiotic (B. infantis+5HMOs) resulted in a significant up-regulation of the anti-viral cytokine IFN-λ at 5 days post infection (peak of viral infection, FIG. 5A) that was associated with a significant reduction in granulocytic cell infiltrates into inflamed lungs as quantified by numbers of lung neutrophils (FIG. 5B) and numbers of lung eosinophils (FIG. 5C), suggesting that early life intervention with the synbiotic (B. infantis+5HMOs) reduces virus-induced severe lung inflammation. These data indicate that early life supplementation with the synbiotic controls effective anti-viral immune responses and promotes rapid resolution of lung inflammation and thus recovery from respiratory infection.

### Pollution-Enhanced Allergic Airway Inflammation Model

Severe RSV infection in early life represents a significant risk factor to develop allergic airway disease later in life. Outdoor air pollution is a major health problem throughout the world. Exposure to particulate matter (PM) has been associated with the exacerbation of several respiratory diseases, including allergic asthma. To mimic such globally relevant conditions, animals recovered from severe PVM infection were sensitized with cockroach allergens in the presence of the pollutant PM2.5 to induce allergic airway inflammation. Such experimental approach allows to test whether nutritional intervention in early life (window of opportunity) does not only alter susceptibility to viral bronchiolitis but may also have sustained immune benefits into adulthood.

Early life respiratory viral infections represent a potential tipping point in the balance between long-term respiratory health and chronic airway disease such as airway allergies. The present study shows that nutritional synbiotic intervention in early life not only reduced severity of airway viral infection but also promoted a sustained immune benefit into adulthood as assessed by reduced susceptibility to pollution-enhanced allergic airway inflammation.

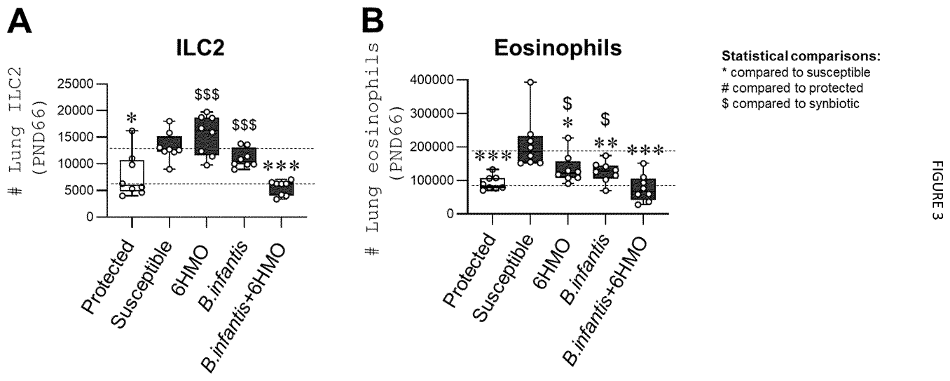

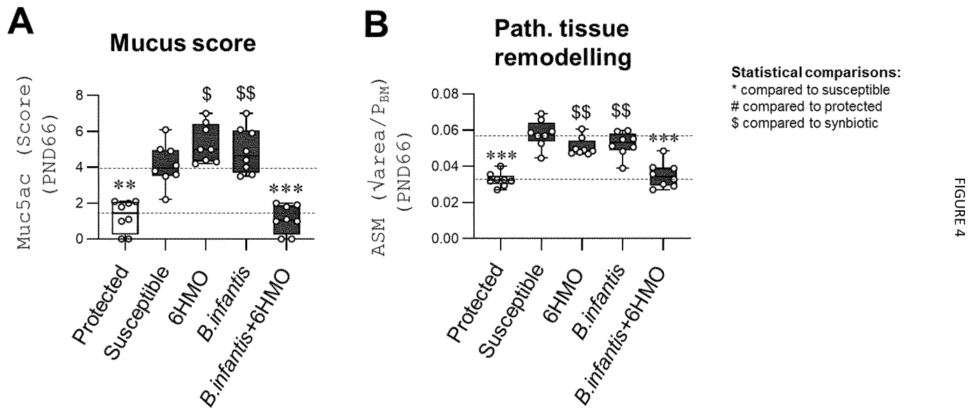

This is exemplified by reduced lung ILC2 (FIG. 3A) and eosinophil numbers (FIG. 3B) as assessed by flow cytometry. Dampened lung inflammation was further associated with a reduced mucus score (FIG. 4A) and improved tissue remodeling (FIG. 4B). Strikingly, and in line with reduced susceptibility to viral airway infection (see FIG. 2), synbiotic intervention (B. infantis+6HMOs) showed superior efficacy over prebiotic (6HMO) or probiotic (B. infantis) interventions alone in reducing pollution-enhanced allergic airway inflammation in adulthood.

### Conclusions

Using a neonatal mouse model of PVM-induced bronchiolitis and early life nutritional interventions, we demonstrate that synbiotic intervention (B. infantis in combination with a mix of human milk oligosaccharides (5HMO or 6HMO)) provides protection from virus-induced bronchiolitis. Importantly, we demonstrate that synbiotic intervention (B. infantis in combination with a mix of human milk oligosaccharides (6HMO)) provides superior efficacy over 6HMOs alone or B. infantis alone to provide protection from virus-induced bronchiolitis. Specifically, synbiotic interventions resulted in a rapid resolution of virus-induced lung inflammation and appropriate lung tissue remodeling upon clearance of the virus.

With the initial host response to respiratory viruses representing a potential tipping point in the balance between long-term respiratory health and chronic airway disease, these findings indicate that early life nutritional intervention with these synbiotics (B. infantis+6HMOs or B. infantis+5HMOs) may represent a preventive strategy to fight severe bronchiolitis in infants and associated risk to develop allergic airway disease at school age. In fact, nutritional synbiotic intervention in early life not only reduced severity of airway viral infection but also promoted a sustained immune benefit into adulthood as assessed by reduced susceptibility to pollution-enhanced allergic airway inflammation. In particular, these results deliver strong evidence that nutritional synbiotic intervention in early life attenuates pathological airway remodeling known to increase the risk of chronic inflammatory diseases such as asthma or COPD later in life.

### Example 2—Ex Vivo Faecal Fermentation Study

### Trial Design

This study was conducted with microbiota colonic incubations as described in Van den Abbeele et al. Frontiers Microbiol. 2023.

Two arms were used in the study. These consisted of:
1. A blend of 7 HMOs: 2FL, 3FL, DFL, LNT, LNnT, 3SL, 6SL
2. 7 HMOs (2FL, 3FL, DFL, LNT, LNnT, 3SL, 6SL)+B. infantis LMG1188

Each arm consisted of six bioreactors (n=6) seeded with the faecal microbiota of respectively 6 individual donors. Donors were 3-month-old infants (±3 weeks) exclusively fed with formulae without HMO/probiotics. The exclusion criteria were antibiotic use in 30 days before sample delivery for the study and previous NEC or gut surgery. This resulted in the enrolment of 6 specific test subjects with an average age of 3.1 (±0.5) months. Intestinal absorption and colonic incubation conditions were then conducted (cf. Van den Abbeele et al. Frontiers Microbiol. 2023).

The blend of 7 HMOs was added at a final concentration of 2.66 g/L. The g/L composition of individual HMOs is described in the table below.

| HMO | Amount (g/L) |
|---|---|
| 2FL | 1.00 |
| 3FL | 0.38 |
| DFL | 0.14 |
| 3SL | 0.10 |
| 6SL | 0.18 |
| LNT | 0.35 |
| LNnT | 0.50 |
| Total | 2.66 |

B. infantis LMG11588 was dosed at a concentration of 5×10⁷ CFU/mL.

Colonic incubations were carried out for 24-hours, with sampling at 0 and 24 hours for:
SCFA levels by HPLC
16S rRNA community profiling of the microbiome

### Results

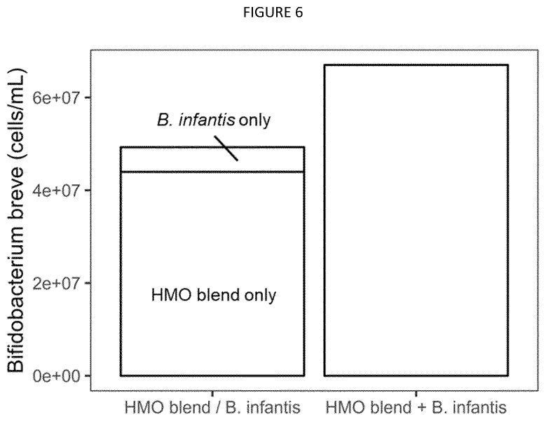

Bifidobacterium breve levels were quantified in liquid after 24 h incubation as described in Van den Abbeele et al., 2023 (DOI: 10.3389/fmicb.2023.1131662). A 7-HMO blend was present as sole carbon source in both conditions. The 6-donor average is plotted. The left bar denotes summed B. breve levels when only a 7-HMO blend is present and B. breve levels when only B. infantis LMG 11588 at 5×10⁷ CFU/mL is present (additive levels). The right bar denotes B. breve levels when the same active ingredients when they are present together (conjunctive levels). The production when no active ingredients is subtracted from each individual condition (blank subtraction). The interaction between 7-HMO and B. infantis LMG 11588 when they are present together results in higher B. breve levels than what could be expected from the summed levels when only a 7-HMO blend and only B. infantis LMG 11588 at 5×10⁷ CFU/mL are present (FIG. 6). Therefore, the data show synergy between the 7-HMOs in combination with B. infantis on the establishment of B. breve within faecal fermentation cultures.

### Conclusions

Using a faecal fermentation study, we demonstrate synergy between the 7-HMOs in combination with B. infantis on the establishment of B. breve.

Alongside B. infantis, Bifidobacterium breve is dominant in the faeces of breast-fed newborns and has a large body of evidence of immune modulating capability (Bozzi Cionci, N., et al., Nutrients, 2018, 10(11)). This evidence includes beneficial modulation of immune responses in the lung, including reduced airways hyperreactivity, T helper type 2 cell activation, eosinophilia and allergen specific antibody production (Hougee, S., et al., Int Arch Allergy Immunol, 2010, 151(2): 107-17).

Thus, the synergistic effect shown here is connected to lung immunity.

All publications mentioned in the above specification are herein incorporated by reference. Various modifications and variations of the described methods and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in molecular biology or related fields are intended to be within the scope of the following claims.

Various preferred features and embodiments of the present invention will now be described with reference to the following numbered paragraphs (para):

1. A composition for use in preventing and/or treating a viral infection in a subject, wherein the composition comprises Bifidobacterium longum subsp. infantis and a HMO mixture consisting of 2′-fucosyllactose (2FL), difucosyllactose (DFL), lacto-N-tetraose (LNT), 6′-sialyllactose (6SL), and 3′-sialyllactose (3SL), and optionally 3-fucosyllactose (3FL) and/or lacto-N-neotetraose (LNnT).

2. A composition for use in promoting a long-term immune benefit in a subject, wherein the composition comprises Bifidobacterium longum subsp. infantis and a HMO mixture consisting of 2′-fucosyllactose (2FL), difucosyllactose (DFL), lacto-N-tetraose (LNT), 6′-sialyllactose (6SL), and 3′-sialyllactose (3SL), and optionally 3-fucosyllactose (3FL) and/or lacto-N-neotetraose (LNnT).

3. A composition for use in i) preventing and/or reducing the risk of allergen sensitisation and/or ii) preventing and/or reducing the risk of developing a respiratory condition in a subject, wherein the composition comprises Bifidobacterium longum subsp. infantis and a HMO mixture consisting of 2′-fucosyllactose (2FL), difucosyllactose (DFL), lacto-N-tetraose (LNT), 6′-sialyllactose (6SL), and 3′-sialyllactose (3SL), and optionally 3-fucosyllactose (3FL) and/or lacto-N-neotetraose (LNnT).

4. A composition for use in preventing and/or reducing the risk of developing asthma in a subject, wherein the composition comprises Bifidobacterium longum subsp. infantis and a HMO mixture consisting of 2′-fucosyllactose (2FL), difucosyllactose (DFL), lacto-N-tetraose (LNT), 6′-sialyllactose (6SL), and 3′-sialyllactose (3SL), and optionally 3-fucosyllactose (3FL) and/or lacto-N-neotetraose (LNnT).

5. The composition for use according to any one of the preceding paras, wherein the HMO mixture consists of 2′-fucosyllactose (2FL), difucosyllactose (DFL), lacto-N-tetraose (LNT), 6′-sialyllactose (6SL), 3′-sialyllactose (3SL) and 3-fucosyllactose (3FL) and/or lacto-N-neotetraose (LNnT).

6. The composition for use according to any one of the preceding paras, wherein the Bifidobacterium longum subsp. infantis is Bifidobacterium longum subsp. infantis LMG 11588 or a strain having an Average Nucleotide Identity (ANI) of at least 99.9% to Bifidobacterium longum subsp. infantis LMG 11588.

7. The composition for use according to any one of the preceding paras, wherein the composition further comprises Bifidobacterium animalis subsp. lactis.

8. The composition for use according to para 7, wherein the Bifidobacterium animalis subsp. lactis is Bifidobacterium animalis subsp. lactis CNCM 1-3446 or a strain having an Average Nucleotide Identity (ANI) of at least 99.9% to Bifidobacterium animalis subsp. lactis CNCM 1-3446.

9. The composition for use according to any one of paras 1-8, wherein the HMO mixture consists essentially of:
i. 31 wt % to 82 wt % of 2FL, preferably 41 wt % to 70 wt %;
ii. 10 wt % to 27 wt % of LNT, preferably 14 wt % to 23 wt %;
iii. 4 wt % to 11 wt % of DFL, preferably 6 wt % to 10 wt %; and
iv. 9 wt % to 34 wt % of 6SL and 3SL combined, preferably 11 wt % to 29 wt %.

10. The composition according to any one of paras 1-8, wherein the HMO mixture consists essentially of:
i. 16 wt % to 69 wt % of 2FL, preferably 22 wt % to 59 wt %;
ii. 9 wt % to 24 wt % of LNT, preferably 12 wt % to 21 wt %;
iii. 2 wt % to 10 wt % of DFL, preferably 3 wt % to 8 wt %;
iv. 8 wt % to 26 wt % of 6SL and 3SL combined, preferably 11 wt % to 22 wt %; and
v. 18 wt % to 50 wt % of 3FL, preferably 11 wt % to 43 wt %.

11. The composition for use according to any one of paras 1-8, wherein the HMO mixture consists essentially of:
i. 34 wt % to 85 wt % of 2FL, preferably 40 wt % to 71 wt %;
ii. 10 wt % to 40 wt % of LNT, preferably 12 wt % to 26 wt %;
iii. 4 wt % to 14 wt % of DFL, preferably 5 wt % to 10 wt %;
iv. 9 wt % to 31 wt % of 6SL and 3SL combined, preferably 10 wt % to 28 wt % and;
v. 6 wt % to 30 wt % of LNnT, preferably 7 wt % to 22 wt %.

12. The composition according to any one of paras 1-8, wherein the HMO mixture consists essentially of:
i. 20 wt % to 60 wt % of 2FL, preferably 22 wt % to 55 wt %;
ii. 4 wt % to 30 wt % of LNT, preferably 6 wt % to 20 wt %;
iii. 1 wt % to 12 wt % of DFL, preferably 2 wt % to 8 wt %;
iv. 7 wt % to 23 wt % of 6SL and 3SL combined, preferably 8 wt % to 22 wt %;
v. 10 wt % to 50 wt % of 3FL, preferably 13 wt % to 46 wt % and
vi. 3 wt % to 25 wt % of LNnT, preferably 5 wt % to 20 wt %.

13. The composition for use according to any one of the preceding paras, wherein the composition is a nutritional composition selected from an infant formula, a starter infant formula, a follow-on or follow-up formula, a baby food, an infant cereal composition, a growing-up-milk, a fortifier such as a human milk fortifier, or a supplement.

14. The composition for use according to any one of the preceding paras, wherein the subject is an infant, a young child or a child.

15. The composition for use according to any one of paras 1 or 5-14, wherein the composition is for use in preventing and/or treating a viral respiratory tract infection.

16. The composition for use according to any one of paras 1 or 5-15, wherein the viral infection causes a disease selected from the group consisting of common cold, influenza (flu), bronchitis, bronchiolitis and pneumonia.

17. The composition for use according to para 16, wherein the disease is bronchiolitis or pneumonia, preferably bronchiolitis.

18. The composition for use according to any one of paras 1 or 5-17, wherein the composition is for use in reducing the risk of developing the viral infection and/or reducing the symptoms associated with the viral infection.

19. The composition for use according to para 18, wherein the symptoms associated with the viral infection are selected from the group consisting of irritation in the lungs, congestion in the lungs, excessive mucus production, fever, cough, wheezing, breathlessness, abdominal cramps, diarrhoea and vomiting.

20. The composition for use according to any one of paras 1 or 5-19, wherein the viral infection is caused by a virus selected from the group consisting of respiratory syncytial virus (RSV), parainfluenza virus (PIV), influenza virus such as influenza virus A (IVA) and/or influenza virus B (IVB), rhinovirus (RV), adenovirus (ADV), metapneumovirus (MPV), bocavirus (BoV), coronavirus (CoV), myxovirus, herpesvirus, enterovirus (EV), and parachovirus (PeV), or any combination thereof.

21. The composition for use according to any one of paras 1 or 5-20, wherein the viral infection is respiratory syncytial virus (RSV) or rhinovirus (RV) infection, preferably RSV infection.

22. The composition for use according to any one of paras 2 or 5-14, wherein promoting a long-term immune benefit in a subject comprises:
i. promoting long-term respiratory health;
ii. preventing and/or reducing the risk of allergen sensitisation; and/or
iii. preventing and/or reducing the risk of developing a respiratory condition later in life.

23. The composition for use according to any one of paras 3, 5-14 or 22, wherein the respiratory condition is a chronic inflammatory disease of the respiratory tract or an allergic respiratory tract disease.

24. The composition for use according to para 23, wherein the chronic inflammatory disease of the respiratory tract is asthma or chronic obstructive pulmonary disease (COPD).

25. The composition for use according to para 23, wherein the allergic respiratory tract disease is recurrent wheeze or asthma, preferably allergic asthma.

26. The composition for use according to any one of paras 3, 5-14 or 22-25, wherein the composition is for use in i) preventing and/or reducing the risk of allergen sensitisation and/or ii) preventing and/or reducing the risk of developing a respiratory condition in the subject later in life.

27. The composition for use according to any one of paras 4-14 or 20-25, wherein the composition is for use in preventing and/or reducing the risk of developing asthma in the subject later in life.

28. The composition for use according to para 26 or para 27, wherein later in life is from 12 months to 12 years after termination of the treatment.

29. A method of:
i) preventing and/or treating a viral infection in a subject;
ii) promoting a long-term immune benefit in a subject;
iii) preventing and/or reducing the risk of allergen sensitisation in a subject;
iv) preventing and/or reducing the risk of developing a respiratory condition in a subject; and/or
v) preventing asthma in a subject,
the method comprising administering to the subject a composition as defined in any one of paras 1 to 28.

1: A method of preventing and/or treating a viral infection in a subject, the method comprising administering a composition to the subject, wherein the composition comprises Bifidobacterium longum subsp. infantis and a HMO mixture consisting of 2′-fucosyllactose (2FL), difucosyllactose (DFL), lacto-N-tetraose (LNT), 6′-sialyllactose (6SL), 3′-sialyllactose (3SL), and 3-fucosyllactose (3FL).

2: A method of preventing and/or reducing a risk of allergen sensitisation in the respiratory tract and/or ii) preventing and/or reducing a risk of developing a respiratory condition in a subject, the method comprising administering a composition to the subject, wherein the composition comprises Bifidobacterium longum subsp. infantis and a HMO mixture consisting of 2′-fucosyllactose (2FL), difucosyllactose (DFL), lacto-N-tetraose (LNT), 6′-sialyllactose (6SL), 3′-sialyllactose (3SL), and 3-fucosyllactose (3FL), and wherein the respiratory condition is a chronic inflammatory disease of the respiratory tract or an allergic respiratory tract disease.

3: A method of preventing and/or reducing a risk of developing asthma in a subject, the method comprising administering a composition to the subject, wherein the composition comprises Bifidobacterium longum subsp. infantis and a HMO mixture consisting of 2′-fucosyllactose (2FL), difucosyllactose (DFL), lacto-N-tetraose (LNT), 6′-sialyllactose (6SL), 3′-sialyllactose (3SL), and 3-fucosyllactose (3FL).

4: The method according to claim 1, wherein the Bifidobacterium longum subsp. infantis is Bifidobacterium longum subsp. infantis LMG 11588 or a strain having an Average Nucleotide Identity (ANI) of at least 99.9% to Bifidobacterium longum subsp. infantis LMG 11588.

5: The method according to claim 1, wherein the HMO mixture consists essentially of:
i. 16 wt % to 69 wt % of the 2FL;
ii. 9 wt % to 24 wt % of the LNT;
iii. 2 wt % to 10 wt % of the DFL;
iv. 8 wt % to 26 wt % of the 6SL and the 3SL combined; and
v. 18 wt % to 50 wt % of the 3FL.

6: The method according to claim 1, wherein the composition is a nutritional composition selected from the group consisting of an infant formula, a starter infant formula, a follow-on or follow-up formula, a baby food, an infant cereal composition, a growing-up-milk, a fortifier such as a human milk fortifier, and a supplement.

7: The method according to claim 1, wherein the subject is an infant, a young child or a child.

8: The method according to claim 1, wherein the composition is administered in an amount effective for reducing a risk of developing the viral infection and/or reducing symptoms associated with the viral infection.

9: The method according to claim 1, wherein the viral infection is caused by a virus selected from the group consisting of respiratory syncytial virus (RSV), parainfluenza virus (PIV), influenza virus (such as influenza virus A (IVA) and/or influenza virus B (IVB)), rhinovirus (RV), adenovirus (ADV), metapneumovirus (MPV), bocavirus (BoV), coronavirus (CoV), myxovirus, herpesvirus, enterovirus (EV), and parachovirus (PeV), and any combination thereof.

10: The method according to claim 9, wherein the viral infection is caused by RSV.

11: The method according to claim 2, wherein the chronic inflammatory disease of the respiratory tract is asthma or chronic obstructive pulmonary disease (COPD).

12: The method according to claim 2, wherein the allergic respiratory tract disease is recurrent wheeze or asthma.

13: The method according to claim 3, wherein the asthma is allergic asthma.

14. (canceled)

15: The method according to claim 1, wherein the HMO mixture consists essentially of:
i. 22 wt % to 59 wt % of the 2FL;
ii. 12 wt % to 21 wt % of the LNT;
iii. 3 wt % to 8 wt % of the DFL;
iv. 11 wt % to 22 wt % of the 6SL and the 3SL combined; and
v. 11 wt % to 43 wt % of the 3FL.

16: The method according to claim 3, wherein the HMO mixture consists essentially of:
i. 22 wt % to 59 wt % of the 2FL;
ii. 12 wt % to 21 wt % of the LNT;
iii. 3 wt % to 8 wt % of the DFL;
iv. 11 wt % to 22 wt % of the 6SL and the 3SL combined; and
v. 11 wt % to 43 wt % of the 3FL.

17: The method according to claim 3, wherein the HMO mixture consists essentially of:
i. 22 wt % to 59 wt % of the 2FL;
ii. 12 wt % to 21 wt % of the LNT;
iii. 3 wt % to 8 wt % of the DFL;
iv. 11 wt % to 22 wt % of the 6SL and the 3SL combined; and
v. 11 wt % to 43 wt % of the 3FL.

18: The method according to claim 2, wherein the subject is an infant, a young child or a child.

19: The method according to claim 3, wherein the subject is an infant, a young child or a child.

20: The method according to claim 2, wherein the composition is a nutritional composition selected from the group consisting of an infant formula, a starter infant formula, a follow-on or follow-up formula, a baby food, an infant cereal composition, a growing-up-milk, a fortifier such as a human milk fortifier, and a supplement.

21: The method according to claim 3, wherein the composition is a nutritional composition selected from the group consisting of an infant formula, a starter infant formula, a follow-on or follow-up formula, a baby food, an infant cereal composition, a growing-up-milk, a fortifier such as a human milk fortifier, and a supplement.
